(19) United States
(12) Patent Application Publication
(10) Pub. No.: US 2008/0146970 A1
(43) Pub. Date: Jun. 19, 2008

(54) GEL DISPENSERS FOR TREATMENT OF SKIN WITH ACOUSTIC ENERGY

(75) Inventors: Peter E. Litman, Wellesley, MA (US); Peter J. Klopotek, Wenham, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: JULIA THERAPEUTICS, LLC, Wellesley, MA (US)

(21) Appl. No.: 11/758,615

(22) Filed: Jun. 5, 2007

Related U.S. Application Data
(63) Continuation-in-part of application No. 11/295,700, filed on Dec. 6, 2005.

(30) Foreign Application Priority Data
Dec. 6, 2006 (US) PCT/US06/46440

(51) Int. Cl. A61N 7/00

(52) U.S. Cl. 601/2

(57) ABSTRACT

Methods and apparatus are disclosed for dispensing fluid in an apparatus for applying acoustic energy to the skin. Acoustic waveguides are disclosed which compensate for distortions that otherwise occur when a focused acoustic beam crosses a boundary, such as the transition from a treatment device to a target region of skin. The invention is especially useful with devices that focus ultrasound energy by condensing a propagating wavefront. The invention compensates for the mismatch in acoustic properties of the device's waveguide and the biological tissue that typically cause portions of the collapsing wavefront to lag behind other portions and, thereby, limit the focusing capabilities of acoustic treatment devices.

### REFERENCE TO RELATED APPLICATIONS

This application is a continuation-in-part of U.S. patent application Ser. No. 11/295,700 filed Dec. 6, 2005, incorporated herein by reference.

This application also claims priority to International Patent Application No. PCTIUS06/46440 filed Dec. 6, 2006, likewise incorporated by reference.

### BACKGROUND OF THE INVENTION

The technical field of this invention is skin treatment and, in particular, the application of acoustic energy to the skin for cosmetic and/or therapeutic purposes.

Human skin is basically composed of three layers. The outer, or visible layer is the stratum corneum. The stratum corneum is essentially a thin layer of dead skin cells that serves, among other things, as a protective layer. Below the stratum corneum is the epidermis layer. The epidermis layer is a cellular structure that forms the outermost living tissue of the skin. Below the epidermis layer is the dermis layer that contains a variety of tissues such as sweat glands, nerves cells, hair follicles, living skin cells, and connective tissue. The connective tissue gives the dermis layer body, shape, and support. Since the epidermis layer lies on top of the dermis layer, the shape, smoothness, and appearance of the epidermis layer is in part determined by the shape of the dermis layer (and largely the connective tissue). Thus, variations in the shape of the connective tissue tend to appear as variations in the epidermis layer. In addition to rhytides of the skin (i.e., skin wrinkles) and, more generally, the skin's texture and elasticity, the dermis layer is also implicated in various other dermatological conditions, such as acne, psoriasis, pigmented lesions, photodamaged skin, stretch marks, and vascular lesions (e.g., spider veins, rosacea, varicose veins, and port wine stains).

There are a number of methods currently being used to treat skin conditions, particularly facial skin wrinkles. Some of these methods include the use of lasers, radio-frequency (RF) ablation, plasma heating, cryo-peeling, chemical-peeling, and dermabrasion. Similarly, optical radiation is currently used to treat unwanted hair, acne and various other condition by delivering energy, typically in the form of heat, to particular regions or biological sites within the epidermis and/or dermis.

However, the various ablation, heating or freezing techniques that presently are practiced can result in significant damage to the epidermis and dermis layers. In some methods, the epidermis layer is peeled or burned away. This presents several problems: opportunistic infections can invade the dermis layer and thus complicate or prolong recovery; the procedure can cause a patient significant discomfort and pain; and the skin can appear raw and damaged for a significant period of time (on the order of weeks or months) while the healing process takes place. All of these side effects are considered undesirable.

Focused acoustic energy, e.g., ultrasound waves, can be a less invasive alternative for treating dermatological conditions. In theory, at least, highly focused acoustic energy can have therapeutic effects at precisely targeted sites with significantly less heating of the biological tissue above and surrounding the target site. However, the use of acoustic energy is often limited by the difficulty in depositing the energy in a tightly focused manner at a target below the skin surface.

A focused acoustic beam typically requires the collapse of a spherical (or cylindrical) wavefront into a point (or a line). While treatment devices with shaped transducers and/or acoustic lenses can be used to concentrate acoustic waves in this manner, the condensing wavefront will be distorted as it cross the boundary from the device to the skin due to the mismatch in acoustic properties of the device's waveguide and the biological tissue. For example, differences in the speed of sound in the waveguide and skin will cause portions of the collapsing wavefront to lag behind other portions and, thereby, limit the focusing capabilities of such acoustic treatment devices.

There exists a need for better devices and methods for the application of acoustic energy to treat dermatological conditions. Devices that can delivery highly concentrated acoustic energy to discrete regions of the epidermis and/or dermis would satisfy a long felt need in the art.

### SUMMARY OF THE INVENTION

Methods and apparatus are disclosed for applying acoustic energy to the skin whereby the wavefront can be controlled to confine the focused energy to a desired subsurface region. Acoustic waveguides are disclosed which compensate for distortions that otherwise occur when a focused acoustic beam crosses a boundary, such as the transition from a treatment device to a target region of skin. The invention is especially useful with devices that focus ultrasound energy by condensing a propagating wavefront. The invention compensates for the mismatch in acoustic properties of the device's waveguide and the biological tissue that typically cause portions of the collapsing wavefront to lag behind other portions and, thereby, limit the focusing capabilities of acoustic treatment devices.

Unless corrected, the acoustical defocus that results from propagation across the skin boundary will cause a reduction of the surface-depth contrast of the acoustical wave intensity. It has been discovered that a sufficiently high contrast between the energy deposited at the skin surface and the energy deposited in the subsurface target region is important to the therapeutic effect and in order to avoid undesired side effects of the sonic irradiation. In one aspect of the invention, methods and apparatus are disclosed to create sufficient surface-depth contrast of the acoustical intensity between the surface of the skin and the intensity at the therapeutic depth inside the skin as to warrant therapeutic effect within the skin and the absence of side effects on the surface of the skin. In certain embodiments, the surface-to-target depth intensity contrast (ratio) is preferably at least about 1:2, more preferably at least about 1:3 or at least about 1:5. For elongated focal regions (e.g., having a length of at least 10 millimeters), the surface-to-target depth intensity contrast (ratio) can be relaxed and is preferably at least about 1:1.2, more preferably at least about 1:1.3 or at least about 1:1.5.

In one application, the invention relates to methods and apparatus for therapeutic treatment of skin using ultrasound. In particular, the present invention relates to reducing rhytides of the skin (i.e., skin wrinkles), especially facial rhytides, and skin rejuvenation, generally, by controlled application of ultrasound energy into the dermis layer. The ultrasound energy triggers a biological response that causes synthesis of new connective tissue in the dermis through activation of fibroblast cells in the dermis without causing or requiring significant irritation or damage to the epidermis. One use of the present invention is to provide a cosmetic improvement in the appearance of the skin meaning that the treated skin surface will have a smoother, rejuvenated appearance. The invention is also useful to treat various other dermatological conditions, such as acne, psoriasis, pigmented lesions, photodamaged skin, stretch marks, and vascular lesions (e.g., spider veins, rosacea, varicose veins, and port wine stains). By providing focused energy to a subsurface region, the present invention provides such therapies with lesser effects on the epidermis layer of the skin.

According to another aspect of the invention, methods are disclosed for skin treatment by applying a focused ultrasound beam to a region of human skin to stimulate or irritate a dermis layer in the region of the skin so as to cause a change in the dermis layer of the skin that results in a change in a smoothness of the epidermis layer of the skin. Additionally, apparatus for rejuvenating human skin is provided, the apparatus comprising an ultrasound transducer, coupled to an ultrasound driver, for propagating ultrasound waves into a region of human skin in response to signals from the ultrasound driver, and a control device constructed and arranged to focus the signals provided by the ultrasound driver circuit to control the ultrasound waves provided by the ultrasound driver so as to stimulate or irritate a dermis layer in the region of the skin to cause a cosmetic improvement in an appearance of the skin.

According to a further aspect of the invention, transducer configurations are disclosed, which are capable of applying focused ultrasound energy to a dermis region of human skin. The transducers can comprise a transducer and an acoustical waveguide disposed adjacent to an ultrasound emitting surface of the transducer, wherein the shape, thickness and composition of the acoustical waveguide determines a depth focus of the ultrasound energy in the skin. Additionally, at least one surface of the waveguide, preferably a skin-contacting surface, can be configured to compensate for the defocusing effects of the mismatch of acoustic properties between the waveguide and the skin.

In one embodiment of the invention, a method of rejuvenating human skin is provided, the method comprising applying a focused ultrasound beam to a region of human skin to generate a shock wave to mechanically disrupt a dermis layer in the region of the skin so as to cause a change in the dermis layer of the skin that results in a change in a smoothness of an epidermis layer of the skin.

In a further aspect of the invention, the acoustic pulses which are used to treat the skin have pressure amplitudes that are sufficiently high to introduce non-linearity, that is to say, the speed of propagation of the pulses through the target region of dermis will be higher than the normal speed of sound propagation through skin. For example, in skin, the normal speed of sound is approximately 1480 m/sec. However, at high enough amplitudes, skin tissue becomes more elastic and the speed of propagation can increase to as high as about 1500 m/sec. The magnitude of this non-linear behavior varies not only with pulse amplitude, but also with the duration of the pulse. Typically, the non-linear behavior will be exhibited, with acoustic pulses having intensity (within the target region) of about 500 to about 1000 watts/cm² and is preferably applied by pulses having durations ranging from about 10 nanoseconds to about 200 microseconds.

In another aspect of the invention, the acoustic pulses that are used to cause therapeutic effects in the skin produce negative pressure in the sub-surface target region, over at least a non-negligible fraction of the acoustical pulse duration. The negative pressure (which can also be considered as tensile stress), at sufficient amplitude and duration, causes tissue to be mechanically stretched or even torn apart. Negative pressure pulses can also trigger cavitation, which causes further mechanical tissue disruption. The gross effects of negative pressure pulses (e.g., on tissue or cellular levels) can be observable under optical microscope. Other effects are also detectable on a nanometer scale (e.g., on a molecular level).

In another aspect of the invention, tissue-disrupting negative pressure can be inherent in the acoustical wave itself or it can be induced in the focal area by a wave with only positive pressure. The second case can be caused by the propagation of the strongly focused acoustical wave in focal region and does not have any simple analog in propagation of electromagnetic radiation. The propagation of an intense and focused acoustical wave in an area of the focus is influenced by nonlinear effects.

One result of this non-linearity is distortion the waveform of the pulses as they travel through the skin, converting waves typically having approximately Gaussian amplitude (pressure) profile to waves that presents a much sharper leading face, essentially a “shock-wave” at the target region below the surface of the skin. In a normal wave propagation mode, there is essentially no net movement of dermal material. However, when acoustic waves exhibit non-linearity, material does move, creating a negative pressure, or vacuum effect, in the tissue in the wake of the pulse. This negative pressure can induce the tissue damage of the present invention, tearing tissue structures apart, heating the region and, thereby, triggering the synthesis of new connective tissue.

In another aspect of the invention, methods and apparatus are disclosed for applying acoustic energy to an elongated region of skin, whereby the wavefront can be controlled to confine the focused energy to a desired elongated subsurface region. In certain embodiments, the sub-surface region of focus can be very elongated. This elongation enables the system to scan a large area of skin economically. This time economy factor, important to the user of the device, will result, however, in a reduction of the surface-to-target depth intensity contrast. For example, in an elongated focusing system that yields a one-dimensional focusing of the wavefront, the surface-to-target depth intensity contrast might be about 1:1.2. An analogous spherical system with two-dimensional focusing might achieve a contrast of 1 to 1.4.

In yet another aspect of the invention, a fluid dispenser can be incorporated in the handpiece (or overall system). For example, a disposal cartridge can be employed to dispense an acoustic gel, or a coolant or a marker or a therapeutic agent. The cartridge can be joined to the handpiece (or system) via a coupling mechanism that facilitates fluid transfer for deliver to the skin. Markers in the form of trace elements or photodetectable substances can be dispensed onto the skin and the system can further include a sensor to detect the marker and then activate the acoustic energy generator.

Those skilled in the art will appreciate that there are two ways to define intensity contrast. One is based on the amplitude of the acoustical field and the other on its energy flow. Unless otherwise noted herein, the term “intensity” is used in its energy flow sense, e.g., power (energy/second) transmitted through a unit area.

In the present invention, the conflict between the desire to have very elongated focal area and having significant surface-target depth intensity contrast is resolved through strong and undistorted focusing. Various methods are disclosed for achieving high contrast by controlling the wavefront such that the wavefront is already convergent inside the acoustical waveguide before the entrance to the skin, and the wavefront inside the skin is strongly convergent and largely free of distortions.

In a further aspect of the invention, strong focusing can have an additional benefit as it reduces the possibility of injury caused by ultrasound energy propagating deep into the body. Some areas of facial skin are very thin, e.g., above the cheek bones or forehead. A high intensity contrast between target depth and underlying tissue or organ depth will reduce the possibility of damage to underlying bone or other tissue structures. For example, for applications such as facial applications where bone lie below a relatively thin skin layer, substantial energy penetration at depths greater than 1 mm should be avoided. In other applications, the depth of sensitive underlying structures may be at depths of 2 mm, 3 mm or even 5 mm. For all of these applications, the deep penetration-to-target depth intensity contrast (ratio) is preferably at least about 1:2, more preferably at least about 1:3 or at least about 1:5. For elongated focal regions (e.g., having a length of at least 10 millimeters), the deep penetration-to-target depth intensity contrast (ratio) can be relaxed and is preferably at least about 1:1.2, more preferably at least about 1:1.3 or at least about 1:1.5.

In yet another aspect of the invention, intensity contrast can be realized through segmentation of the transducer into two or more generally symmetrical parts located approximately on a spherical (or cylindrical) perimeter and aligned is such a way that the resulting additive contribution of this segments creates a wavefront with high intensity contrast. In one embodiment, a monolithic cylindrical transducer can be used with a central area that is acoustically or electrically blocked. More generally, such device designs can create a large synthetic aperture from two or more small aperture emitters. Because the emitters will each individually have a small aperture, the distortion of the wavefront can be much less pronounced.

In yet another aspect of the invention, the transducer's surface interface to the skin can be designed to be substantially flat. The flatness provides easy and efficient acoustical transmission from waveguide to the skin. Alternatively, grooved surfaces can be employed, with the groove depth preferable less than 500 micrometers, more preferably less than about 100 micrometers.

The invention is particularly useful for reducing the appearance of human skin wrinkles. Embodiments of the present invention can provide a smoother, rejuvenated appearance of the skin, without adversely damaging the epidermis layer of the skin.

### BRIEF DESCRIPTION OF THE DRAWINGS

In the drawings, which are incorporated herein by reference and in which like elements have been given like reference characters,

FIG. 1 illustrates one embodiment of an ultrasound generating apparatus according to the invention;

FIG. 1A illustrates one embodiment of a handpiece according to the invention;

FIG. 1B illustrates another embodiment of a handpiece according to the invention;

FIG. 2 illustrates one embodiment of an ultrasound transducer that can be used in the invention;

FIG. 3 illustrates another embodiment of a transducer that can be used in the invention;

FIG. 4 illustrates another embodiment of a transducer that can be used in the invention;

FIG. 5 illustrates another embodiment of a transducer that can be used in the invention;

FIG. 6 illustrates a control system that can be used to control the apparatus illustrated in FIG. 1;

FIG. 7 illustrates another embodiment of a transducer that can be used in the invention;

FIG. 8 illustrates a pattern of ultrasound application over a region of skin in accordance with one aspect of the invention;

FIG. 9 illustrates a pattern of ultrasound application over a region of skin in accordance with another aspect of the invention;

FIG. 10 is a schematic illustration of another ultrasound applicator;

FIG. 11 is a schematic illustration of an alternative embodiment of an ultrasound applicator;

FIG. 12 is a schematic illustration of another embodiment of an ultrasound applicator;

FIG. 13 is a schematic illustration of a further embodiment of an ultrasound applicator;

FIG. 14 is a schematic illustration of yet another ultrasound applicator embodiment;

FIG. 15 is a graph of an illustrative excitation waveform for driving a transducer according to the invention;

FIG. 16 is a graphic simulation of a acoustic wave generated by a transducer driven by the waveform of FIG. 15;

FIG. 17 is a schematic illustration of hemi-spherical (cylindrical) transducer design according to the invention;

FIG. 17A is a simulated wavefront analysis for an uncompensated acoustic pulse from a transducer, such as shown in FIG. 17, passing across a skin boundary;

FIG. 17B is a simulated ray-tracing analysis for an uncompensated acoustic pulse from a transducer, such as shown in FIG. 17, similarly passing across a skin boundary;

FIG. 18 is a schematic illustration of a wavefront compensating element (an acoustic surface lens structure) for use with a spherical (or cylindrical) transducer to compensate for skin boundary distortions;

FIG. 18A is a wavefront analysis for a compensated acoustic pulse from a wavefront compensating element, such as shown in FIG. 18, passing across a skin boundary;

FIG. 18B is a ray-tracing analysis for a compensated acoustic pulse from a wavefront compensating element, such as shown in FIG. 18, similarly passing across a skin boundary;

FIG. 19 is a schematic illustration of another wavefront compensating element (an acoustic surface lens structure with a filler material) for use with a spherical transducer to compensate for skin boundary distortions and further showing a wavefront analysis for a compensated acoustic pulse therefrom passing across a skin boundary;

FIG. 20 is a schematic illustration of yet another embodiment of the invention employing an aspheric transducer together with a wavefront analysis for an acoustic pulse generated by such an aspheric transducer passing across a skin boundary;

FIG. 21 is a further wavefront analysis of an acoustic pulse from an aspheric transducer, showing the contributions from different regions of the transducer;

FIG. 22 is a further wavefront analysis of two acoustic pulses generated simultaneously by two separated, pie-shaped, transducers; and

FIG. 23 is a more generalized schematic illustration of a boundary-compensating, segmented, transducer design according to the invention.

FIG. 24 is a schematic illustration of a further embodiment of an ultrasound applicator.

FIG. 25 is a schematic illustration of an alternative embodiment of an ultrasound applicator.

FIG. 26 is a schematic illustration of another embodiment of an ultrasound applicator

FIG. 27 is a schematic illustration of another alternative embodiment of an ultrasound applicator.

FIG. 28 is a schematic illustration of a further embodiment of an ultrasound applicator.

FIG. 29 is a schematic illustration of an alternative embodiment of an ultrasound applicator.

FIG. 30 is a schematic illustration of yet another embodiment of an ultrasound applicator.

FIG. 31 is a schematic of an alternative embodiment of an ultrasound applicator.

FIG. 32A is a simulated ray-tracing analysis for an acoustic pulse from a transducer.

FIG. 32B is another simulated ray-tracing analysis for an acoustic pulse from a transducer.

### DETAILED DESCRIPTION

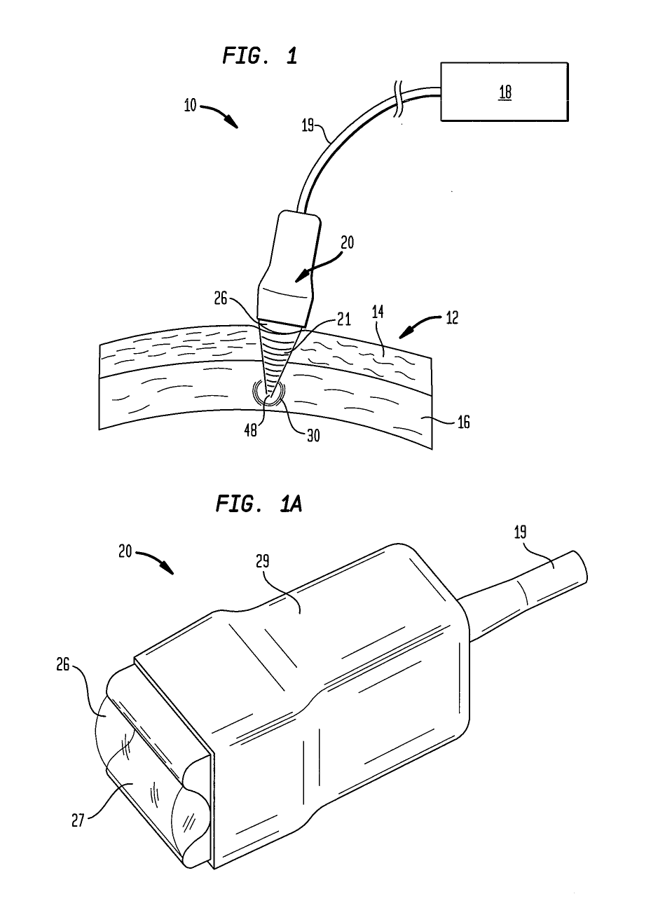

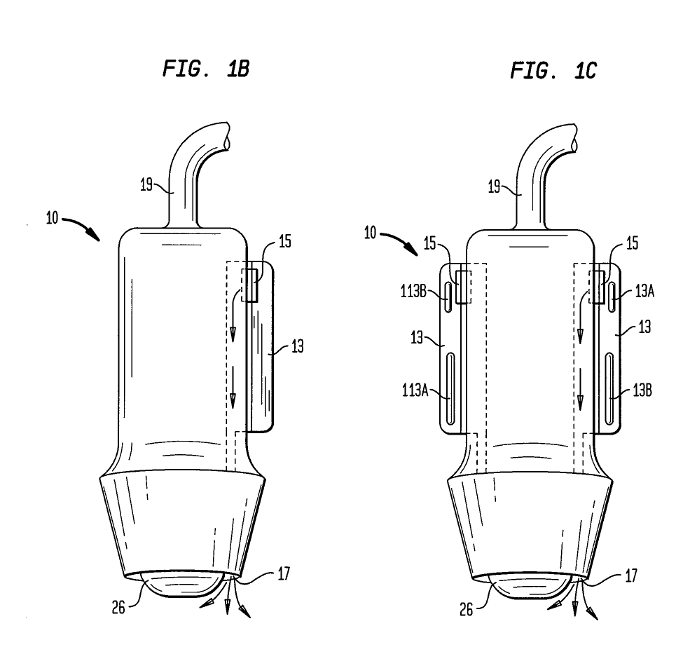

FIG. 1 generally illustrates an ultrasound generating apparatus 10 that can be used to apply controlled, localized, focused ultrasound to a region of human skin. The apparatus includes a control circuit 18 coupled to handpiece 20 via electrical means 19 which can be a cable or the like. The handpiece includes one or more transducer elements as will be described in more detail below. In response to control signals from control circuit 18, the handpiece 22 generates ultrasound waves 21. Handpiece 22 can have one or more elements, such as piezoelectric elements, that actually produce the ultrasound or similar acoustic waves as well as one or more focusing elements. The handpiece also includes an acoustically-transmitting waveguide 26 having a skin contacting surface, although in some applications it can be desirable to employ an acoustical coupling medium, such as a biocompatible hydrogel between the surface of the waveguide and the skin. Even when a gel is employed, the waveguide surface proximal to the skin should be understood to be a skin contacting surface.

Apparatus 10 is used to direct the ultrasound waves 21 into skin 12. The ultrasound waves are focused so that the focused ultrasound waves create a region of stimulation and/or irritation 30 in the dermis layer 16 of skin 12. Focusing ultrasound waves 21 within region 30 allows localized enhancement of the fluence of the ultrasound beam directed into the skin in region 30. This allows part of the energy in ultrasound waves 21 to be absorbed in region 30. This results in stimulation and/or irritation of region 30. Since the region 30 is principally contained in dermis layer 16, there should be little, if any, significant adverse damage to the epidermis layer 14.

In FIG. 1A, handpiece 20 is further illustrated having a body 29 and an acoustic waveguide 26 with a skin-contacting surface 27. As shown in FIG. 1A, and discussed further below, waveguide 26 can be generally cylindrical (semi-spherical) with a surface 27 that is aspheric to compensate for boundary-induced defocusing effects.

In FIG. 1B, another embodiment of a handpiece 10 is shown including a disposal cartridge 13 that can be employed to dispense an acoustic gel, or a coolant or a marker or a therapeutic agent. The cartridge 13 can be joined to the handpiece via coupling mechanism 15 that facilitates fluid transfer for delivery to the skin (e.g., via a peripheral port 17). The cartridge 13 can be removable to facilitate cleaning of the handpiece, or replacement of cartridges. The cartridge 13 can be removable from the handpiece separately from the other fluid dispensing components (e.g. 15, 17).

In FIG. 1C, another embodiment of a handpiece 10 is shown including a fluid dispenser consisting of a plurality of cartridges 13 that can be deployed to dispense an acoustic gel, or a coolant or a marker or a therapeutic agent. The plurality of cartridges 13 can be joined to the handpiece via coupling mechanism 15 that facilitates fluid transfer for delivery to the skin (e.g., via a peripheral port 17). The plurality of cartridges 13 may be symmetrically placed (as shown in FIG. 1C). Again, each cartridge 13 can be removable from the handpiece separately from the other fluid dispensing components (e.g. 15, 17). In additiona, each cartridge 13 may be disposable.

As shown in FIG. 1C, each cartridge can contain a plurality of fluid reservoirs 113A, 113B. Each reservoir 113A, 113B is joined to the handpiece via coupling mechanism 15 that facilitates fluid transfer for delivery to the skin. Each reservoir can contain a different fluid or fluid component to allow control of fluid component ratios.

Markers in the form of trace elements or photodetectable substances can be dispensed onto the skin and the system can further include a sensor (as discussed below) to detect the marker and then activate the acoustic energy generator. The apparatus can be further constructed such that the system does not operate unless the handpiece and cartridge are properly locked or otherwise joined together by coupling mechanism 15.

The disposable cartridge, or plurality thereof, can be coupled to the handpiece via coupling mechanism 15, which preferably provides a leakage-free conduit between a fluid-containing chamber in the cartridge and a fluid distributor within the handpiece. The coupling mechanism can also provide a sensor for confirming the contents and/or for alerting the user to impending exhaustion of the cartridge contents. The fluid to be dispensed can be an acoustic coupling gel, a chilled liquid or gas (e.g., to cool the epidermis) or a cosmeceutical (e.g., containing Vitamin C or hyaluronic acid).

Although shown in connection with a disposable cartridge, it should be clear that the invention also encompasses the delivery of such fluids from a base station, e.g., via connection 19 or a separate conduit (not shown). A control switch can be incorporated into the disposable cartridge, handpiece or otherwise form part of the system to control flow of the fluid. The cartridge 13 can further include a source of propellant or the system can rely upon pressure or pump elements incorporated into the handpiece 10 or base station 18 (as shown in FIG. 1). The control switch can be manually operated, thereby allowing the user to control the flow of the fluid. The control switch can also be responsive to a sensor incorporated into the disposable cartridge, handpiece or otherwise forming part of the system to control flow of the fluid. The sensor can be responsive to motion of the handpiece. In combination with, or alternative to, a motion sensor, a sensor can measure a parameter related (e.g. proportionally) to the amount of fluid dispensed. Such parameters can be, for example, electrical conductivity (e.g., resistivity, capacitance, and/or impedance), optical reflection or ultrasound reflection. As mentioned above, the fluid can contain trace elements or photodetectable substances to allow a sensor to determine the thickness or amount of fluid dispensed by measurement of one or more of the aforementioned parameters. Based upon an output from a sensor the apparatus can dispense additional fluid, activate an alarm to alert the user to a lack of fluid, or deactivate the apparatus.

In one embodiment, signals from the sensor can be used to determine whether enough gel or other topical agent has been applied to the skin and alert the user (e.g., by visual or audible signal) of suboptimal performance, or in the case of dangerous situations, automatically provide a shut-off signal to a controller. Sensors can also be used to determine to determine whether the gel or other agent is has been contaminated or is otherwise unsuitable for use in the apparatus.

The term “ultrasound” as used in this disclosure is intended to encompass both conventional “ultrasound” as typically used to describe high-frequency acoustic waves up to about 100 megahertz and “hypersound” as typically used to describe very high frequency acoustic waves greater than about 100 megahertz. In general, “ultrasound” is used within this disclosure to describe acoustic waves capable of inducing controlled hyperthermia or cavitation in skin tissue, or pulsed waves having an amplitude large enough to induce shock waves or tissue stretching (tensile pressure) in the skin tissue. Hyperthermia, as used in this disclosure, is a condition in which an elevated temperature is induced in a region of the body for therapeutic purposes.

As noted, a feature of the invention lies in providing a focused ultrasound beam that irritates and/or stimulates the dermis layer of the skin without significant or detrimental irritation of the epidermis layer and/or other tissue at depth beyond the designated treatment depth. Focusing of the ultrasound beam can be provided by one or more focusing elements. In certain embodiments of the invention, relatively low power ultrasound is used to gently stimulate and/or irritate the dermis to induce a biological response that results in synthesis or production of new connective tissue over a period of time that extends beyond the time of application of the ultrasound energy. Treatment by the present invention can be a one-time therapy or can entail multiple treatments extending over a relatively long period of time, such as days, weeks, or months, in order to stimulate the body to produce new connective tissue in the dermis layer.

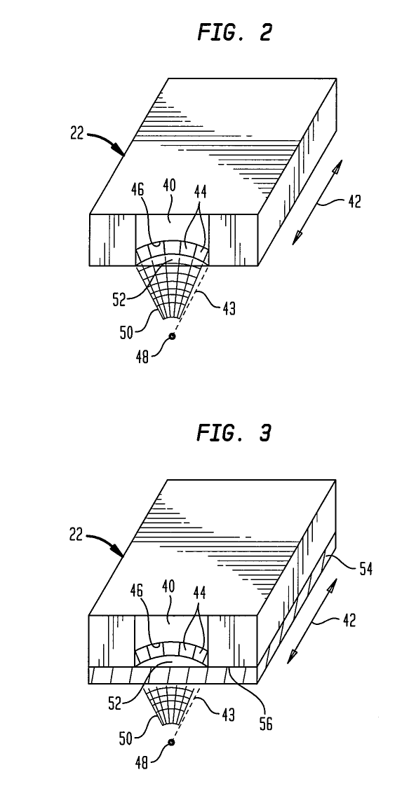

FIG. 2 illustrates one embodiment of a transducer 22 for providing a focused ultrasound beam. In FIG. 2, transducer 22 has a concave or cylindrical surface 40 that extends along dimension 42. A number of elongated transducer elements, such as piezoelectric elements 44 are disposed along a surface 46 of region 40. One skilled in the art will appreciate that a single curved transducer or multiple transducer elements could be used in transducer 22. Elements 44 extend longitudinally along the direction of dimension 42. Since elements 44 are disposed along the concave surface, they will transmit the ultrasound beams that they respectively generate towards a focal point 48 that lies at the intersection of the various radii 50 that extend from transducer elements 44 to focal point 48. Thus, by adjusting the radius of curvature of surface 46, the location of focal point 48 can be changed. One skilled in the art will appreciate that in the cylindrical embodiment shown in FIG. 2, focal point 48 extends longitudinally along the direction of dimension 42 to create a scanline 43.

FIG. 3 illustrates another transducer configuration that can be used in accordance with the present invention. In FIG. 3, transducer 22 is fitted with an acoustical waveguide 54 that covers a surface 56 of the transducer. Acoustical waveguide 54 is analogous to acoustical waveguide 26 illustrated in FIG. 1. An acoustical coupling medium, preferably of a material having the same or similar transmissive properties as acoustical waveguide 54 can fill the entire cavity 52. Alternatively, acoustical waveguide 54 can be a single piece that additionally fills cavity 52. The transducer illustrated in FIG. 3 performs in the same manner as transducer 22 illustrated in FIG. 2 however, the addition of acoustical waveguide 54 can make the transducer easier to scan across flat skin surfaces. In addition, acoustical waveguide 54, since it acts to direct the ultrasound waves along the direction of radii 50, can reduce the size and bulk of transducer 22. That is, the addition of acoustical waveguide 54 can allow the radius of curvature of surface 46 to be larger than what would otherwise be required, without waveguide 54, for a given location of focal point 48. Thus, this particular configuration of ultrasound transducer 22 can be easier to manufacture than one having its radius of curvature determined only by the location of focal point 48. This configuration is also useful when higher ultrasound beam intensities are being used because it can prevent overheating of the transducer since the transducer can be made physically larger to better dissipate heat.

In the present invention, the depth of focus of scanline 43 is very close to the surface of the skin, therefore, acoustical waveguide 54 can be used to determine the depth of focus. Acoustical waveguide 54 can be of differing thickness where each different thickness provides a different depth of focus. Use of acoustical waveguides of differing thickness provides a convenient means for changing the depth of focus which can be advantageous in the case where treatment is carried out in, for example, a doctor's office.

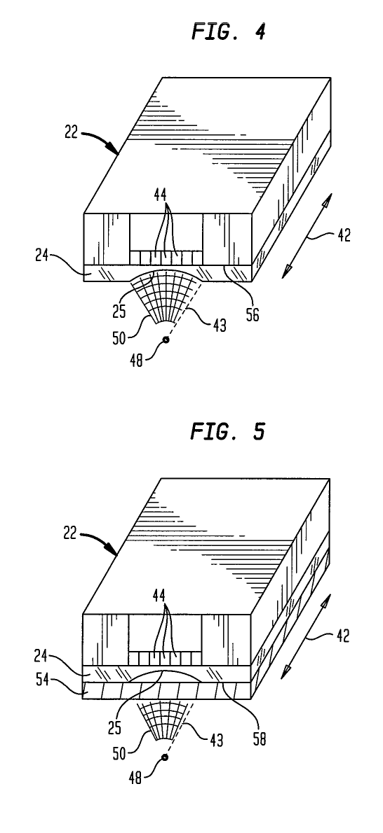

FIG. 4 illustrates another transducer configuration that can be used in accordance with the present invention. In FIG. 4, transducer 22 has a flat or planar configuration and transducer elements 44 are disposed in an essentially planar fashion. A lens 24 having a focusing portion 25 is disposed along the lower surface 56 of the transducer. Focusing section 25, which is cylindrical and extends along the direction 42, acts to focus the ultrasound wave generated by transducer elements 44 along the direction of lines 50 so that the ultrasound waves produced by transducer elements 44 are focused at focal point 48.

FIG. 5 illustrates another transducer configuration that can be used in accordance with the present invention. In FIG. 5, transducer 22 is fitted with an acoustical waveguide 54 disposed at the lower surface 58 of lens 24. Acoustical waveguide 54, in the same manner described in connection with FIG. 3, allows the radius of curvature of focusing section 25 of lens 24 to have a larger radius of curvature than would otherwise be required for a given location of focal point 48. Thus, this particular configuration of ultrasound transducer 22 can be easier to manufacture.

The systems illustrated in FIGS. 1, 2, 3, 4, and 5 are preferably adapted to strongly focus the ultrasound beam, e.g., by focusing with a numerical aperture greater than about 0.3. As illustrated in the figures, the lens preferably has a generally cylindrical geometry to focus the acoustic energy into a line rather than a point.

One skilled in the art will also appreciate that a biocompatible hydrogel can be placed between the skin surface and the lens 24 (in the case of FIG. 4) or acoustical wave guide 54 (in the case of FIG. 5).

One skilled in the art will appreciate that although particular transducer configurations have been illustrated in FIGS. 1-5, a variety of other transducer configurations can be used in the present invention. In addition, a phased array ultrasound transducer can be used. A phased array can be advantageous in that it can be used to focus the ultrasound beam generated by each respective transducer element at a desired focal point depth and location. In addition to focusing the ultrasound beam, the phased array can be used to scan the ultrasound beam over the area of skin to be treated.

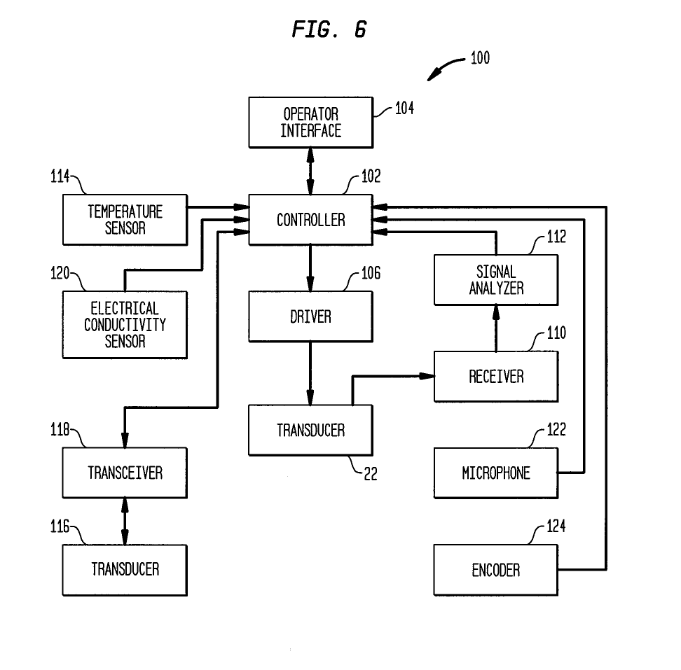

FIG. 6 illustrates a control system that can be used in the present invention to control the amount of energy provided to region 30 of dermis layer 16. The control system 100 includes a controller 102. Controller 102 can include a computer and associated peripherals such as memory and mass storage devices. An operator interface 104, which can include at least a keyboard and display device, allows the user to set various parameters such as the focal point depth, the magnitude of the ultrasound beam to be applied, the duration that the ultrasound beam will be applied, and so on. Control signals from controller 102 are sent to a driver 106. Driver 106 contains means, such as circuitry, such as needed to cause the transducer element or elements of transducer 22 to generate ultrasonic waves.

Control system 100 thereafter includes five different feedback systems that can be used to control the dose of ultrasound energy applied to a patient's skin. One skilled in the art will appreciate that the five feedback systems can be used individually or in any combination.

The first feedback system includes a receiver 110 and a signal analyzer 112. Receiver 110 and signal analyzer 112 can be used to measure the magnitude of the ultrasound energy being applied to the patient's skin and to provide a feedback signal to controller 102 to automatically, or allow the operator to manually adjust the magnitude of the ultrasound beam being delivered by transducer 22.

The second feedback system includes a temperature sensor 114 that can be used to measure the temperature of the skin in the region where the ultrasound energy is being applied. Using temperature sensing as a feedback mechanism can be effective because the surface of the skin where temperature sensor 114 would be located is in close proximity to the region of the skin being heated by ultrasound energy. The sensed temperature reading can then be used by controller 102 to automatically, or manually, under control of the operator, to control the magnitude of ultrasound energy being delivered to the patient's skin by transducer 22.

The third feedback system includes a second ultrasound transducer 116 and transceiver 118. Transceiver 118 and transducer 116 can be used to provide a low level ultrasound signal that can be used for diagnostic and feedback purposes to controller 102. Transceiver 118 and transducer 116 can also be used as an echo-locating system for target location. That is, the low power ultrasound signals can be used to locate microorgans, such as hair follicles, in the skin to aid in treatment.

Furthermore, if driver 106 is replaced with a transceiver or if an additional receiver is provided and connected to transducer 22 and controller 102 then the echo-locating function can be performed using one transducer. That is the transducer 22 can be placed on the patient's skin and, under control of controller 102, low power ultrasound waves can be used for target location and placement. Once a location for treatment has been established, controller 102 can be switched to a treatment mode and a higher power ultrasound wave can then be applied using transducer 22 to treat the skin.

More generally, the low power ultrasound can be used to locate a condition below the epidermis that causes an irregularity in the smoothness of the epidermis. Higher power ultrasound can then be used to treat the area.

Furthermore, the low power ultrasound signal can also be used to automatically determine the depth of focus for the ultrasound energy. For example, the low power or diagnostic ultrasound signal can be used to locate the depth of the interface between the dermis and the epidermis in the area to be treated. The depth of focus for the high power or therapeutic ultrasound can then be set based on this measurement to ensure that the ultrasound energy is focused in the dermis layer.

The fourth feedback system includes an electrical conductivity sensor 120 that can be used to measure the electrical conductivity of the patient's skin in the region where the ultrasound energy is being applied. The degree of electrical conductivity sensed by sensor 120 can then be used by controller 102 to automatically, or manually, under control of the operator, control the magnitude of ultrasound energy being delivered to the patient's skin by transducer 108.

The fifth feedback system includes a broadband microphone 122 connected to controller 102. When cavitation is used as a mechanism to provide dermal irritation, microphone 122 can be placed on or near the skin in the region being treated. The collapse of a bubble created by application of ultrasound in the dermis creates a characteristic acoustic signature that is detected by microphone 122. The signal provided by microphone 122 can then be used by controller 102 with appropriate signal processing to control the ultrasound energy provided by transducer 22. The user can also listen to the signal provided by microphone 122 and manually control the ultrasound energy.

Controller 102 should be programmed so that transducer 22 delivers a spatially uniform ultrasound dosage in the area of the skin that is being treated to ensure uniform stimulation of the dermis layer. The method of the invention appears to be most effective when there is, on average, a homogeneous deposition of energy in the region of the skin that is being treated.

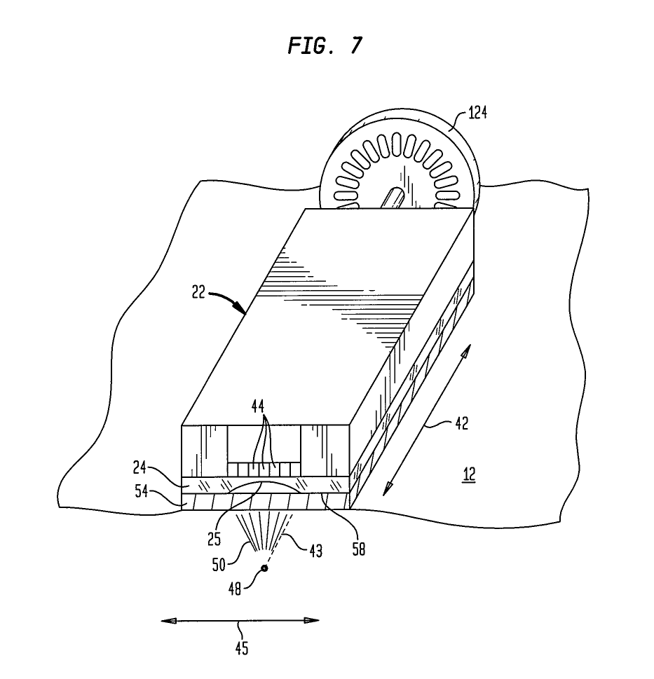

Referring to FIG. 7 transducer 22 is illustrated as being scanned along a direction defined by double-headed arrow 45. While transducer 22 is being scanned along the direction of arrow 45, it is delivering an ultrasound beam focused at a focal point or depth 48 in the dermis layer of the skin. Focal point 48 extends longitudinally along the direction of dimension 42 to create a scanline 43. Controller 102 therefore needs to be programmed to deliver a uniform level of energy in two dimensions; one along the direction or dimension 42 and one in a direction of scanning along line or dimension 45.

The energy delivered by transducer 22 into the skin can be delivered in a continuous manner or in discrete increments. One skilled in the art will appreciate that the ultrasound energy can be continuous in one dimension for example, dimension 42 and discrete in another dimension, for example dimension 45 or vice versa. One skilled in the art will appreciate that the ultrasound energy can be delivered continuously in both dimensions or discretely in both dimensions.

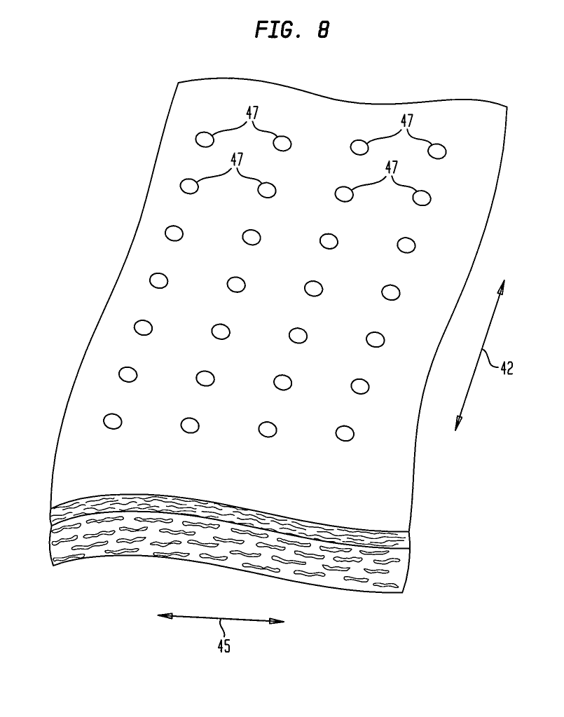

If the ultrasound energy is delivered discretely in both dimensions 42 and 45, then a pattern of ultrasound energy application such as illustrated in FIG. 8 results where each point 47 represents a location where ultrasound energy has been applied. If the ultrasound energy is applied in a manner that is continuous in both dimensions 42 and 45, then the area in between points 47 would also have ultrasound energy applied thereto.

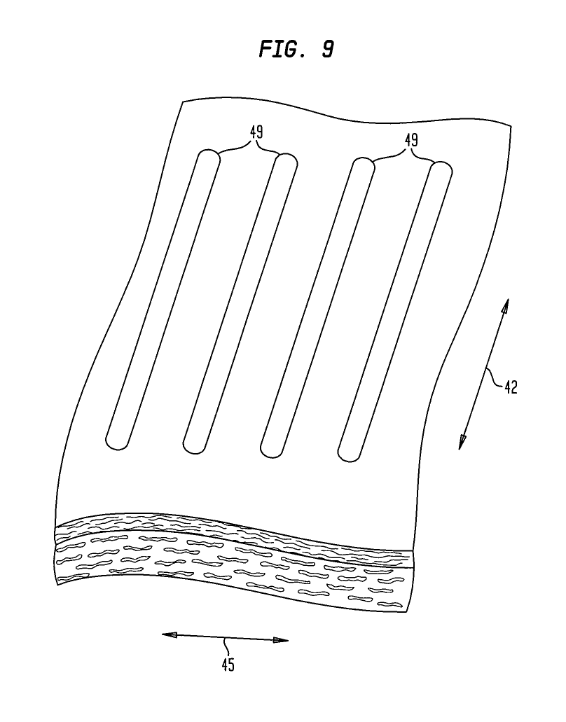

If the ultrasound energy is delivered discretely in dimension 45 and continuously in dimension 42, then a pattern of ultrasound energy application such as illustrated in FIG. 9 results where regions 49 represent regions where ultrasound energy has been applied.

In the case of continuous ultrasound application, both the speed of scanning along direction 45 and the power being applied must be controlled simultaneously. In the same manner, if discrete application of ultrasound energy is being used, then the distance between points 47 along the direction of arrow 45, the speed with which transducer 22 is moved along the direction of arrow 45, and the timing of individual energy deposition must be controlled to provide homogeneous exposure.

As illustrated in FIGS. 6 and 7 an encoder 124 can be provided. Encoder 124 can be, for example, a wheel that rolls along the skin as the transducer is scanned across the skin. An electrical signal which can be analog or digital in nature, is then provided to controller 102. Controller 102 uses the signal from encoder 124 to determine the speed with which transducer 22 is being scanned across the skin surface and the distance being traveled. With this information, controller 102 can be programmed to adjust the ultrasound pulse frequency and intensity of the ultrasound energy in relation to the scanning speed and distance traveled to achieve, on average, spatially uniform ultrasound dosage if discrete ultrasound pulses are being used. In the same manner, if continuous power is being used, then controller 102 will adjust the ultrasound beam energy in relation to scanning speed to achieve a homogeneous application of ultrasound energy in the target area.

In another embodiment, an acoustically transparent plate can be placed on the skin over the area to be treated and then transducer 22 and encoder 124 are then scanned across the acoustically transparent plate. Scanning the transducer across the plate can also provide a way of delimiting the area to be treated to avoid over-treating or under-treating the area of the skin.

To use the method and apparatus of the invention to reduce or eliminate human skin wrinkles, a physician or technician (“the user”) sets a desired depth of the focal point for the ultrasound beam so that the ultrasonic energy is substantially concentrated in the dermis layer of the skin. This depth is typically in the range of five microns to five millimeters. The magnitude of the ultrasound energy to be deposited in the dermis layer in also determined. The duration of treatment and the volume of the dermis layer to be stimulated and/or irritated determine the power level necessary.

The frequency of the ultrasound beam is also chosen. The ultrasound wave frequency should be within the range between approximately 1 megahertz and 500 megahertz. Preferably, the ultrasound beam frequency is relatively low frequency ultrasound between the range of approximately 3 and 80 megahertz and, more preferably in some applications between about 10 and 80 megahertz. The ultrasound beam frequency chosen is based upon a consideration of the depth of penetration of a given ultrasound frequency wave into the skin and the power required to cause an appropriate stimulation and/or irritation of the dermis region of interest.

Obviously, the above-described steps can be performed in any order.

Once these parameters have been set, the ultrasound transducer is then scanned over the wrinkle area of the skin. Typically, an area much larger than or extending significantly beyond the area occupied by the wrinkle is subjected to the ultrasound beam. Preferably, to be effective, the area of the skin that is subjected to treatment is on the order of ten times larger than the area of the wrinkle itself.

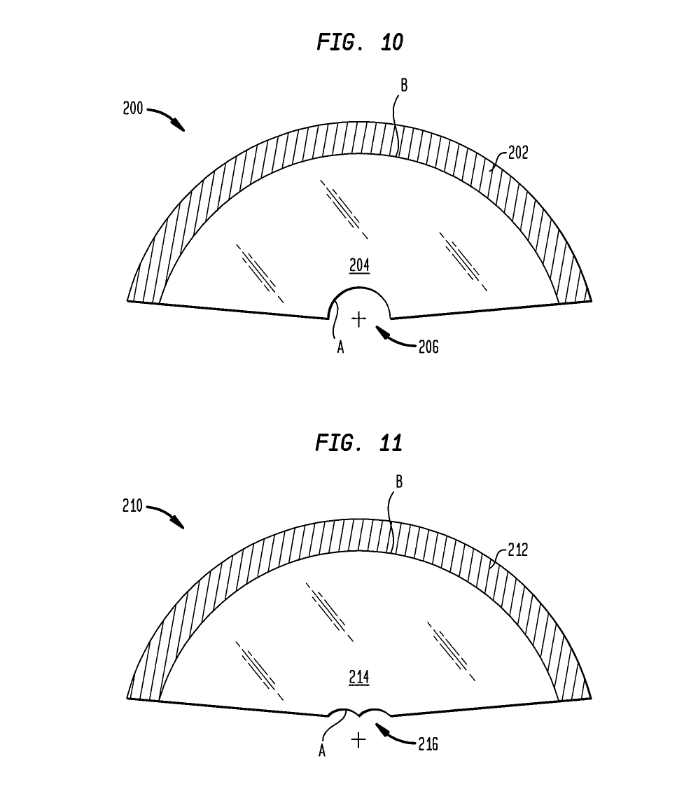

In a further aspect of the invention, structures are disclosed that compensate for distortions that can arise as focused acoustic energy travels from the device to a target region below the skin surface. Accordingly, boundary-compensating acoustic waveguides are disclosed. The terms “boundary compensating element” and “boundary compensating surface” are used herein to describe the general class of devices or structures that can compensate for distortions in energy or wavefront propagation that otherwise occur when a focused acoustic beam crosses a boundary, such as the transition from a treatment device to a target region of skin. For example, the boundary compensating element can be an acoustic lens or waveguide (or a waveguide surface structure) that selectively modifies portions of a passing acoustic wavefront. Such modifications of the wavefront can be achieved by the shape or by variations in the thickness or material composition of the boundary compensating element. FIGS. 10-14 illustrate various apparatus according to the invention that incorporate one or more boundary compensating elements.

For skin treatments with acoustic energy, good quality acoustical focus and high focusing angle (high NA) are typically required at a depth of few hundred micrometers. A particularly useful mode for delivering such energy to a subsurface target region is to employ a convergent wavefront that is semi-spherical. (The term “semi-spherical” is used herein to describe a 3-dimensional shape that is spherical in one axis, e.g., a cylindrical geometry. Spherical, semi-spherical, cylindrical, hemi-cylindrical and semi-cylindrical are also used interchangeably to describe the shape of wavefronts that useful in providing focused acoustic energy.)

While a collapsing semi-spherical wavefront will produce a good focus as it propagates through a material of uniform acoustic properties, any distortions encountered during convergence will produce a less localized (more spatially dispersed) region of high intensity and a lower peak negative pressure.

Generally speaking, the radius of curvature of the wavefront defines the depth of the focus. The shape of the acoustical wavefront at the origin is typically controlled by the shape of the transducer, e.g., a monolithic or semi-monolithic curved transducer. If no waveguide is interposed between the transducer and the skin, the skin would need to occupy the cavity formed by the shaped transducer. When a high focusing angle is needed, working without a waveguide would require forcing the skin into a cylindrical hemi-spherical concave transducer on the order of 300 micrometer radius - allowing only a small region of skin to be treated for each placement of the instrument. Moreover, this would be practical only if the groove is filled entirely with skin tissue (or with a fluid that transmits sound at the same speed as sound propagates through tissue.

The term “waveguide” is intended encompass any acoustically transmissive (transparent) structure positioned between a transducer and a target, e.g., a subsurface skin region. The principal acoustic properties of waveguide material(s) for the purposes of this disclosure are: the speed of sound, acoustical impedance, acoustical absorption coefficient and its frequency dispersion (dispersivity). The term “wavefront” can encompass a single wavefront, a series of wavefronts at the same frequencies (with matched or unmatched phases) or a sum of wavefronts at different frequencies, each frequency having its own phase.

The term “aspheric” is intended to encompass shapes, typically curved shapes, that are not spherical in nature, i.e., shapes that cannot be characterized by a single radius of curvature. As noted above, sphericity and asphericity are usually used in the one-dimensional context (e.g., a cylindrical or hemispherical focus). Thus, the terms “spheric” and “aspheric” are shorthand expressions that should be read to encompass hemi-spheric and hemi-aspheric shapes, as a matter of course. The term “DFHS” as used herein refers to “deviation from hemi-sphericity” and can be measured in terms of local distortion in radius of the wavefront curvature.

The terms “compensating,” “boundary compensating,” and “wavefront compensating,” as well as similar phrases, are used herein to describe modifications that alter a wavefront (or begin with a specifically designed wavefront) to reduce the defocusing effects of passage across a transmission boundary (from one medium to another having different acoustic properties). Thus, the term “compensating” should be read to encompass systems, methods and designs that prevent and/or reduce such defocusing.

With reference to FIGS. 10-14, the geometry of a collapsing wavefront (in quasi-monolitic transducer systems) is largely defined by the geometry of the interfaces and the speed of sound in the waveguide material(s) and in the tissue. The principal interfaces are (a) the interface between the waveguide and the skin, (b) the interface between the transducer and the waveguide, and (c) the interface between two or more waveguide materials (having different acoustical wave propagation speeds). The wavefront geometry can also be influenced, to a lesser extent, by the dispersive properties of the waveguide material(s) and diffraction. If diffractive effects are ignored, ray tracing approximations can help illustrate the present invention.

In the illustrative figures, the transmission of the sound through the waveguide can be defined (or modeled) by the acoustical absorption coefficient(s) of the waveguide material(s), the distance that the waves transverse inside the waveguide material(s) and by the phenomenon of reflection of acoustical waves at the interfaces. By proper choice of waveguide materials and the dimensions of the waveguide, acoustic absorption will contribute less to the losses of acoustical energy than the reflection at the interfaces. The amount of reflected energy is largely a function of differences in acoustical impedance of the materials on both sides of each interface and the angles of incidence of the waves at each location along the interfaces. In other words, losses of acoustical signal are governed mainly by the reflections at interfaces due to a mismatch of acoustical properties, and angle of incidence. The numerical aperture (NA) in the skin and in the waveguide can be assumed to be the same. The term “numerical aperture” or “NA” is intended to encompass not only the precise scientific definitions (e.g., the product of the index of refraction in the object space multiplied by the sine of half the angular aperture of the lens or lens equivalent structure) but also more generally is used herein to simply describe the angle of focus.

In FIG. 10 a treatment device 200 is shown including curved transducer 202 and waveguide 204, whereby a non-distorted hemi-spherical wavefront is generated at (and equal in geometry to) interface B. Skin tissue is assumed to entirely fill cavity 206. The hemi-spherical interface's B center of curvature is exactly at the focal point marked with a small cross. During the propagation from B to A the acoustical wave preserve's the hemi-sphericity of the wavefront and location of its focal point. The interface A is semi-spherical as well. Interface's A center of curvature is located exactly in the same position as the curvature's center of interface B. This is a “monocentric” design. The design causes undesirable difficulty in pressing skin into longitudinal hemi-spherical trench. In terms of asphericity, the structure of FIG. 10 can be defined as follows:

DFHS(B)=0

DFHS(A)=0

In FIG. 11 another embodiment of the present invention is illustrated by a device 210, having curved transducer 212 and waveguide 214 similar to those described above. Again, transducer 212 is configured to generate a non-distorted hemi-spherical wavefront (DFHS(B)=0) at interface B (and equal in geometry to it). The hemi-spherical interface's B center of curvature is above the focal point marked with a small cross. During the propagation from B to A the acoustical wave preserves its hemi-sphericity. The interface A is specially designed to transform the incoming hemi-spherical wavefront into another wavefront which is hemi-spherical as well, however with a different curvature and different position of the center. The new curvature center marked with the small cross now becomes the new focal point. The location of the new focal point is shifted away from the center of curvature defined by the curved transducer and interface B. This is no longer a mono-centric design. Its advantage lies in much easer access of skin into a shallower, aspheric groove 216. The NA in the skin is reduced as compared to the waveguide. The case can summarized in following way:

DFHS(B)=0

DFHS(A)=0

such that DFHS(B)+DFHS(A)=0

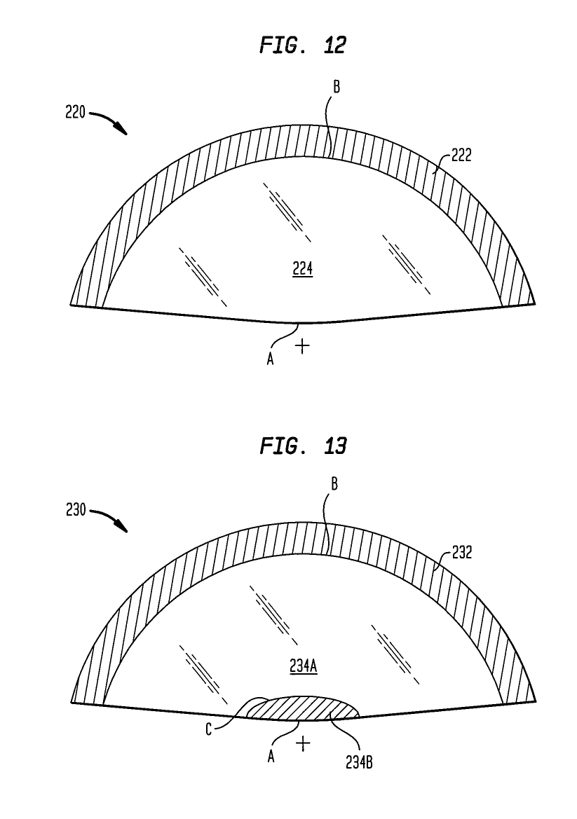

In FIG. 12, another embodiment of the present invention is illustrated by a device 220, again having curved transducer 222 and waveguide 224 similar to those described above. However, transducer 222 presents an intentionally distorted NON-hemi-spherical shape at the interface B thereby adapted to introduce a non hemi-spherical wavefront into the waveguide 224. The distortion of the non-hemi-spherical wavefront is compensated at the interface A, so that, when in the skin, the restored hemi-spherical wavefront is well focused. The cross again indicates the location of the focal point. The case is summarized in the following way:

DFHS(B)+DFHS(A)=0

The obvious advantage of this design lies in smooth access of the skin to the interface A. In some applications is it desirable that the interface A preferably be flat or only mildly curved. However, the advantages of this design, in some instances, will be offset by greater difficulty in manufacturing of the transducer-waveguide interface B.

In FIG. 13, yet another embodiment of the present invention is illustrated by a device 230, having curved transducer 232 similar to those described above, together with a two-part waveguide 234A and 234B. Again, transducer 232 is configured to generate a non-distorted hemi-spherical wavefront (DFHS(B)=0) at interface B (and equal in geometry to it). Between materials 234A and 234B, which have two different sound propagation speeds, is located interface C. The interface C distorts the wavefront in such a way that it compensates the distortion in interface A. Interface A can be flat or mildly curved and is easy accessible to the skin. Interfaces A and B are now easy to manufacture. Interestingly, interface C can be (but not need to be in general case), approximately of semi-spherical shape. In such a case however the location of the center of the curvature of C should be different than the center of curvature of B. (A non semi-spherical shape does not have center of curvature at all). The NA in the skin can be either reduced or enlarged as compared to NA at the interface B. This embodiment can be summarized as follows:

DFHS(B)=0 and

DFHS(A)+DFHS(C)=0

In the embodiments of FIGS. 10-13 one common feature is that the center of the curvature B is at least in proximity of the focal point. Such structures are made from few components and have few interfaces. The complexity of such interface shapes is, however, balanced by the fact that the transducer at the interface B is at least approximately hemi-spherical.

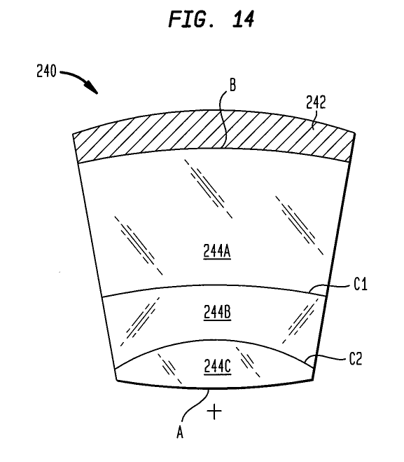

In FIG. 14 another embodiment of the invention is illustrated, in which a flat transducer design can be utilized. In FIG. 14, a device 240 is shown, having a transducer 242 similar to those described above, together with a three-part waveguide 244A, 244B and 244C. In the embodiment of FIG. 14, the center of curvature of B is very distant from the location of the focus. In extreme case center of B can lay even in infinity, and B would be, in such a case, totally flat. Although illustrated with a three-part waveguide, it should be clear that similar structures can be assembled now from even more interfaces and more materials. Its appearance and theory of operation thus becomes analog to the design of optical microscopes. Depends on the choice of materials, the signs of the interface curvatures can be even reversed.

In some designs, the waveguide materials can be metals (like Al or Ti), as the curved interfaces of such materials have powerful focusing abilities (because the difference in the speed of sound on both sides of the interface can be very large). The NA in the skin is enlarged as compared to the NA at the interface B and the case can be summarized is as follows:

DFHS(A)+DFHS(B)+DFHS(C1)+DFHS(C2)+contribution from other interfaces=0

Various materials can be used to construct the waveguides useful in the present invention. On illustrative material is TPX® polymer (available, for example, from Mitsui Chemicals, Inc.), a 4-methylpentene-1 based polyolefin with good acoustic transparency as well as heat and chemical resistant qualities. Alternatively, Rexolite® plastic (available, for example, from C-Lec Plastics, Inc.), a cross linked polystyrene plastic produced by cross linking polystyrene with divinylbenzene, can be employed. Yet another useful material is Kynar®, a polyvinyl difluoride (PVDF) polymer (available, for example, from Arkema Group, SA). In some applications, aluminum or other metals can be used, particularly with ceramic transducers. Although metals exhibit a larger impedance mismatch (and, hence, greater reflection losses), the greater focusing power of such materials can be advantageous in particular applications.

Although the biological mechanism is not completely understood, it appears that hyperthermia and/or cavitation, either alone or in combination, can cause a biological response. It appears that denaturation by hyperthermia of at least some of the intracellular proteins, intercellular proteins, and/or enzymes induces a biological or healing response in the body. The biological response results in the synthesis of new connective tissue by fibroblast cells in the dermis in addition to the preexisting connective tissue. The new connective tissue fills out the skin. It is the process of adding new connective tissue to the dermis layer that causes reduction in the appearance of skin wrinkles and improved shape, smoothness, and appearance of the skin.

One mechanism by which the biological response can be stimulated is through hyperthermia. The amount of energy deposited using hyperthermia is typically that required to raise the temperature of the dermis layer to somewhere is the range of 47° C. to 75° C. Preferably, the temperature of the dermis layer that is being treated is increased to between approximately 55° C. and approximately 65° C.

These ranges are selected so as to denature a relatively small fraction of the proteins in the dermis. At a temperature of approximately 47° C., it takes several tens of seconds to denature a small fraction of the proteins in the dermis. By contrast, at a temperature of 73° C., the same small fraction of the proteins in the dermis are denatured in several tens of microseconds. One skilled in the art will appreciate that there is a trade off between exposure time and the amount of energy being applied. The higher the level of energy to be applied, the lower the required exposure time and vice verse. Elevating the dermis layer to a temperature in approximately the range from 55° C. to 65° C. appears to provide a workable compromise between the length of time for the treatment and the amount of energy to be imparted to the skin.

Another mechanism by which a biological response can be induced is cavitation. Preferably, when using cavitation alone or in combination with hyperthermia, enough energy needs to be applied to the dermis to generate, in the dermis, a cavitational bubble. When the bubble collapses, a shock wave results that mechanically, in as localized area, tears apart tissue in the dermis causing dermal inflammation or irritation and a resultant biological response. The biological response results in the synthesis of new connective tissue.

Another mechanism by which a biological response can be induced in through the use of pulsed acoustic waves. Pulsed acoustic waves having sufficient amplitude can be used to create a negative pressure wave at the focal point so as to induce a shock wave type response in the dermis. As with the collapse of the cavitational bubble, the shock wave mechanically, in a localized area, tears apart tissue in the dermis causing a dermal irritation and a resultant biological response. The biological response results in the synthesis of new connective tissue.

It will be appreciated that the magnitude of energy deposited in the skin as a function of the frequency of the ultrasound wave, the time the ultrasound wave is applied, the area of the skin that is treated, thermal diffusion of the heat in the skin, and the impedance of the skin to ultrasound energy can be varied to provide the desired biological response. The present invention typically uses dosages that are significantly lower than conventional hyperthermia therapies. For example, at the surface of the epidermis, the intensity of the ultrasonic waves can be in the range of approximately 100 to 500 watts/cm². At the focal point in the dermis layer, under some conditions, the intensity of the ultrasonic waves can be in the range of approximately 500 to 1500 watts/cm². The electrical impulse that drives such transducers can be a simple square wave or step function or a gated oscillatory driver. More preferably, the impulse can be designed to maximize the application of negative pressure to the target tissue.

In certain preferred embodiments, it can be desirable to transmit the acoustic energy to the target region as a “monopulse,” that is a single pulse with little or no “ringing.” A monopulse can be useful in enhancing negative pressure or “cavitation” effects, whereby a high degree of tensile force is experienced by the target tissue. Stressing tissue in this manner is believed to cause microvoids in the dermal tissue structure and, thereby, trigger biological responses. In other applications a swinging or ringing pulse can be advantageous. Preferably, regardless of the nature of the pulse, the time duration of such pulses (measured, for example, by the full-width, half value (FWHV) of the monopulse or the FWHV of a single oscillation in a “ringing” pulse train) is less than about 5000 nanoseconds, more preferably, less than 500 nanoseconds, for example on the order of 100 nanoseconds for monopulses.

The duration of a single oscillation acoustic pulse is largely a function of the acoustical thickness of the transducer and is essentially the time of acoustical roundtrip in the transducer thickness the thickness of the transducer. Plastic foil transducers, for example, can be used and preferably have a thickness less than about 500 micrometers, more preferably, less than 200 micrometers, for example on the order of 100 micrometers, if monopulses are desired. Ceramic transducers are preferably less 1 millimeter in thickness, more preferably ranging from about 200 micrometers to about 500 micrometers.

The duration of a ringing pulse train is however not a function of the acoustical roundtrip but rather is dependant either on the ringing characteristics of the transducer or on the design of its electrical energizer. In case of the step function electrical driver, the pulse duration is function of the mechanical Q in the ringing, which in turn depends on material internal acoustical loses and the degree of the acoustical mismatch between the acoustical impedance of the transducer material and the acoustical impedance of the waveguide. The bigger the mismatch the longer the ringing. In case of driver supplying gated oscillations at the frequency of the transducer's ringing, the duration of the pulse is controlled by and equal to the gating.

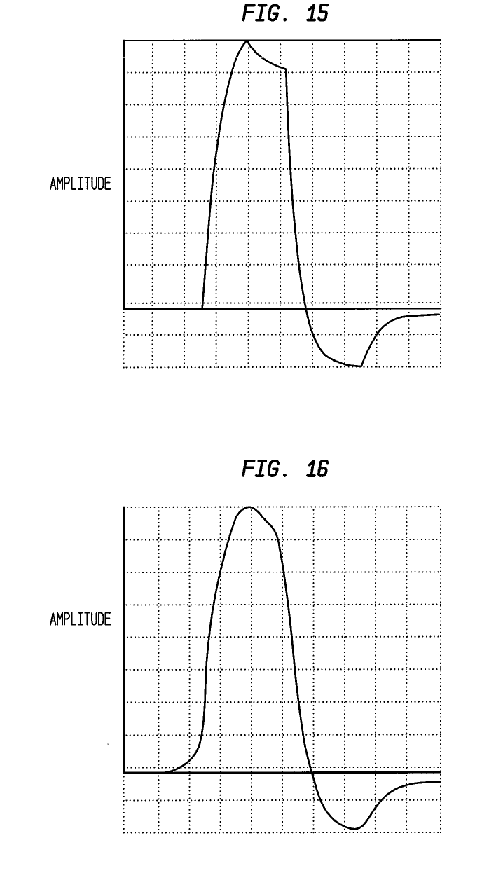

FIG. 15 and FIG. 16 are graphic simulations of acoustical pulse pressures (effectively with reversed amplitudes, i.e., the negative values have been here “repoled”) that can be generated according to the invention based on a simple step function excitation voltage. This excitation will drive the transducer to make a single expansion or contraction of the thickness. Such acoustic responses can be achieved in transducers of either ceramic or piezoelectric composition. (For ringing pulse trains, oscillating transducers made from ceramic piezoelectric materials may be preferred.) FIGS. 15 and 16 illustrate the same numerically modeled pressure waveform in two locations. FIG. 15 simulates an acoustic pulse as initially generated in the vicinity of the transducer, while FIG. 16 illustrates the acoustic pulse after it has propagated for a short distance.

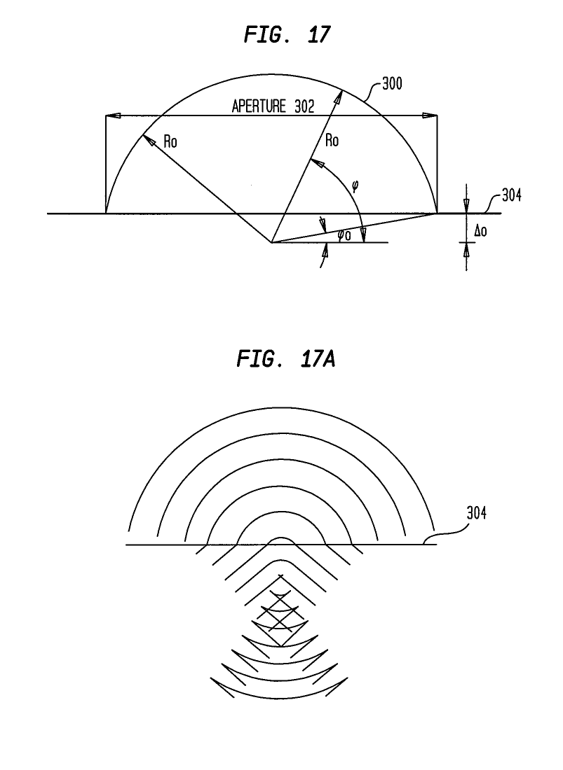

FIG. 17 is a schematic illustration of spherical transducer design according to the invention. The transducer 22 is of generally spherical (cylindrical form) and generates a spherical wavefront. The last undisturbed wavefront 300 is shown. The acoustically active aperture 302 of the interface 304 can be, for example, about 1.2 mm. In the illustrated design, Φ₀ can be set to be about 10 degrees.

FIG. 17A is a simulated wavefront analysis for an uncompensated acoustic pulse from a transducer, such as shown in FIG. 17, passing across a skin boundary 304. The illustration in FIG. 17A presents isochronal lines that is a series of instantaneous snapshots of a single pulse taken at different time or at different phase. As can be seen, the wavefront is disturbed, crossing the interface 304 and, consequently, poorly focused.

FIG. 17B is an alternative illustration in the form of a simulated ray-tracing analysis for the same uncompensated acoustic pulse from a transducer, such as shown in FIG. 17, passing across the interface 304 (e.g., the skin boundary), again showing the poorly organized focus.

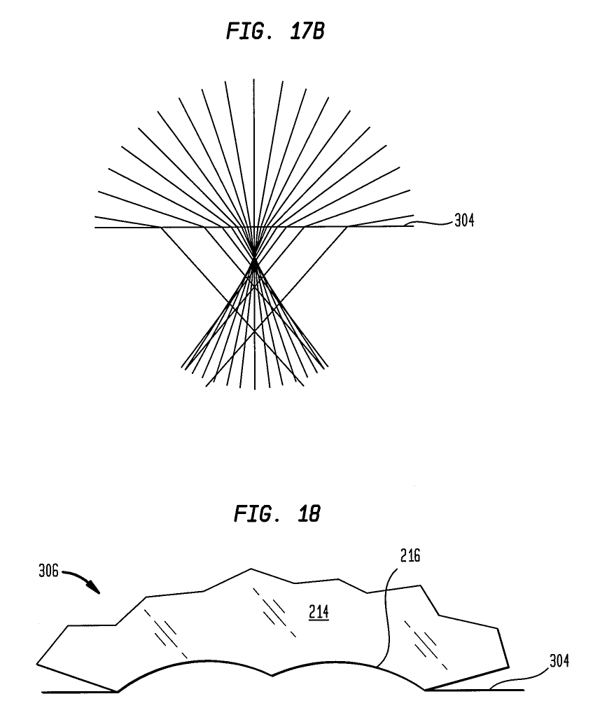

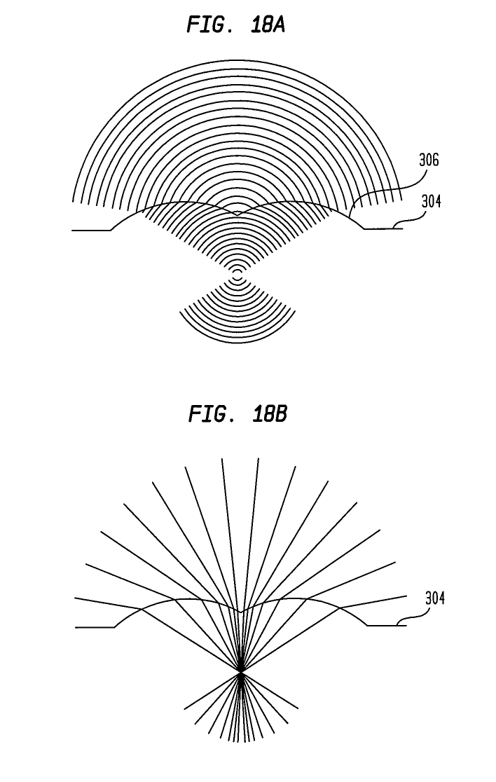

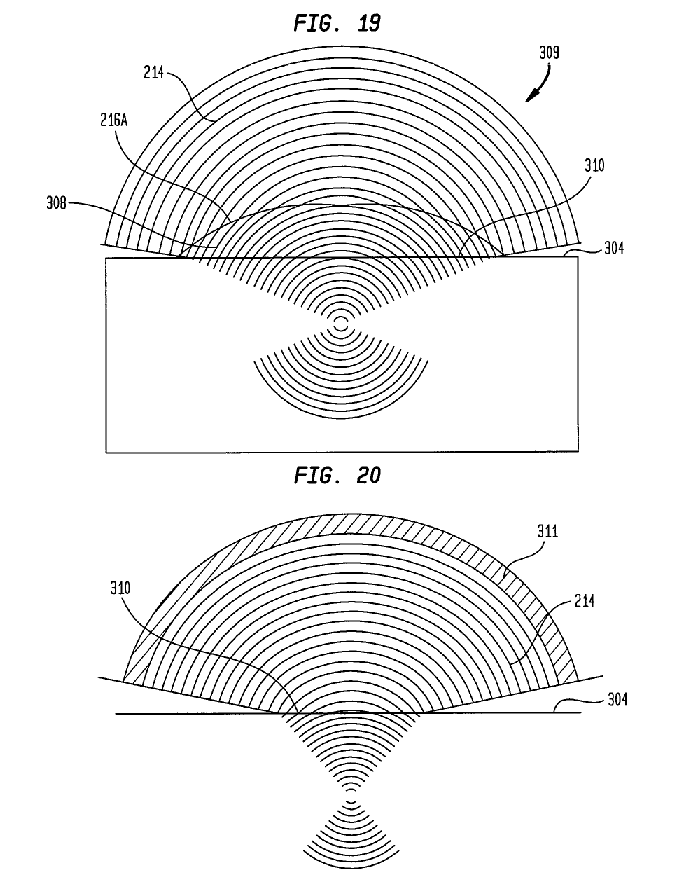

FIG. 18 is a schematic illustration of a wavefront transforming element 306 (an acoustic lens structure 214 with a shaped skin contacting surface element 216) for use with a spherical transducer, such as shown schematically in FIG. 17. Element 306 is designed to preserve hemi-spherical nature of the wavefront at the skin boundary as discussed above. The element 216, in essence, transforms the wavefront's radius of the curvature, shifting its focal point deeper into the tissue. Thus element 306 compensates for the boundary condition at the skin interface 304, which would otherwise lead to defocusing effects. Because of the surface shape of element 306, the wavefront before and after crossing the skin boundary remains cleanly hemi-spherical. Only the focal point and radius of curvature are transformed.

FIG. 18A is a simulated wavefront analysis for a hemi-spherical acoustic pulse from a transducer, such as shown in FIG. 17, coupled to the transforming element 306 of FIG. 18, again illustrating passage of a wavefront across a skin boundary. As can be seen, the wavefront is redirected by element 306 to achieve a deeper focus.

FIG. 18B is an alternative illustration (in the form of a ray-tracing analysis) for the same compensated acoustic pulses from a spherical or hemispherical transducer, passing across the interface 304 (e.g., the skin boundary), again showing the well defined focus.

FIG. 19 is a schematic illustration of another wavefront compensating assembly 309 (an acoustic lens structure 214 with a shaped skin surface element 216A and a filler material 308), again for use with a spherical transducer to compensate for skin boundary distortions. The filler material should have acoustic properties (e.g., speed of sound propagation) that are closely matched to those of biological tissue. Examples of such materials are bakelites (phenol formaldehyde resins), ethylvinylacetates, polyurethanes, polystyrols, polyethylenes (TCI polyethylenes, in particular) aqualenes and butadiene polyurethanes. Those skilled in the art will appreciate that this list is merely illustrative and other materials are also readily available and useful as filler materials. By employing the filler material and an internal compensating structure, a flat contacting surface 310 can be preserved. FIG. 19 further shows a wavefront analysis for a compensated acoustic pulse passing across a skin boundary and achieving a tight focus.

FIG. 20 is a schematic illustration of yet another embodiment of the invention employing an aspheric transducer 311 as a wavefront compensating element. In this embodiment, the wavefront propagating through the waveguide 214 is not spherical but becomes spherical as it passes across the skin boundary 304, as illustrated by the wavefront analysis in the figure. By employing the aspheric transducer, a flat contacting surface 310 can again be preserved.

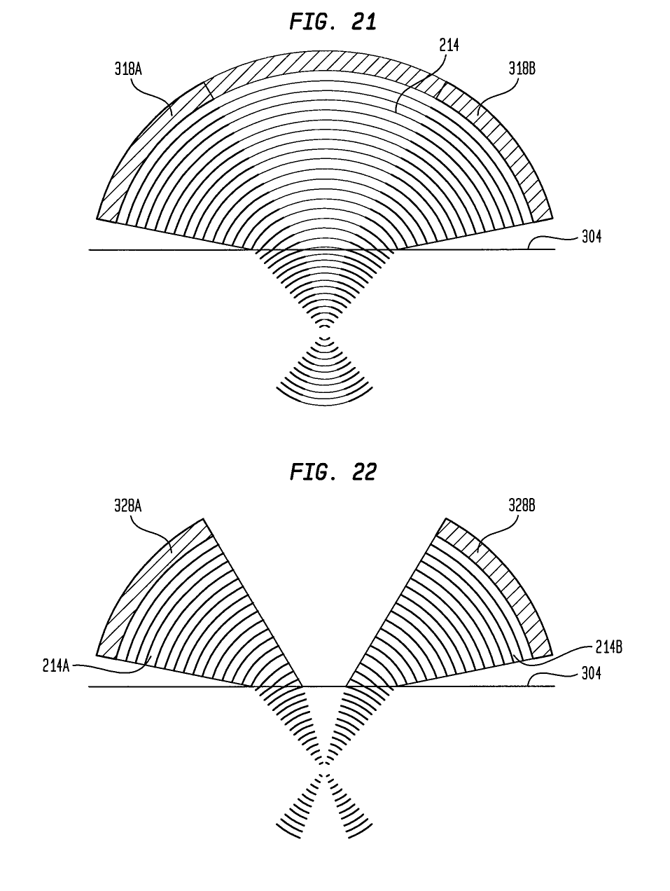

FIGS. 21 and 22 illustrate a further feature of aspheric transducers. FIG. 21 presents a further wavefront analysis of an acoustic pulse from an aspheric transducer, showing the contributions from different regions of the transducer. Thus the contributions from transducer regions 318A and 318B are cumulative but do not require a continuous unitary transducer. Thus, as shown in FIG. 22, the same effect can be achieved by two independent but synchronized transducers 328A and 328B. Thus, FIG. 22 provides a further wavefront analysis of two acoustic pulses generated simultaneously by the two separated transducer segments 328A and 328B. As can be seen, the segmented wavefronts are redirected to a common focus.

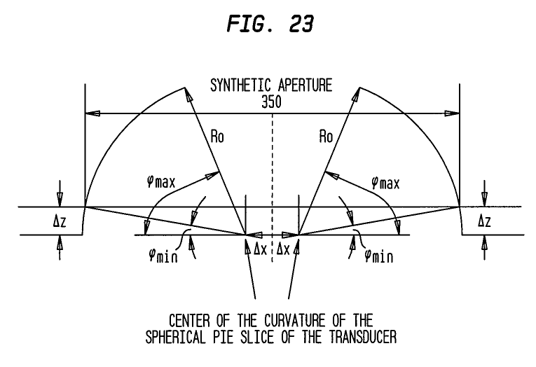

FIG. 23 is a more generalized schematic illustration of a boundary-compensating, segmented, transducer design according to the invention. By proper spacing of the segments 328A and 328B (shown in FIG. 22), a synthetic aperture 350 can be defined. Depending upon the application the segments also need not be aspheric. By proper, choice of the transducer shape, subtended angle (Φ) and/or offset (Δx), two nominal foci (in the absence of a boundary condition) can be superimposed into a single focus. In other words, the transducer segments need not share a common center of curvature. By proper choice of the offset, the segmented transducer can compensate for the acoustic mismatch waveguide and target tissue. Although this embodiment is illustrated with two transducer elements, it should be clear that more than two segments can also be employed and, in some instances, it may also be desirable to modify the radius of curvature for one or more transducer segments as well as, or alternatively to, providing a offset.

While segmented transducer approaches may not provide optimal wavefront focusing, they nonetheless can achieve fairly good compensation for distortions to a wavefront in tissue and be easier and/or more economical to manufacture, especially when ceramic transducers are employed. Moreover, this approach has an additional advantage in that acoustic energy can be blocked from entering the skin at angles that approach vertical, thereby further reducing the possible of excessive energy penetrating deep into the skin and possible having adverse effects on tissue or bone structures that lie beneath the dermis. In certain applications, it can be advantageous to block most or all of the energy from entering the skin either vertically or at angles less than 30 degrees from vertical (i.e. a 60 degree shield), or preferably at angles less that 20 degrees from vertical, or more preferably in some instances at angles less than 10 degrees or 5 degrees from vertical.

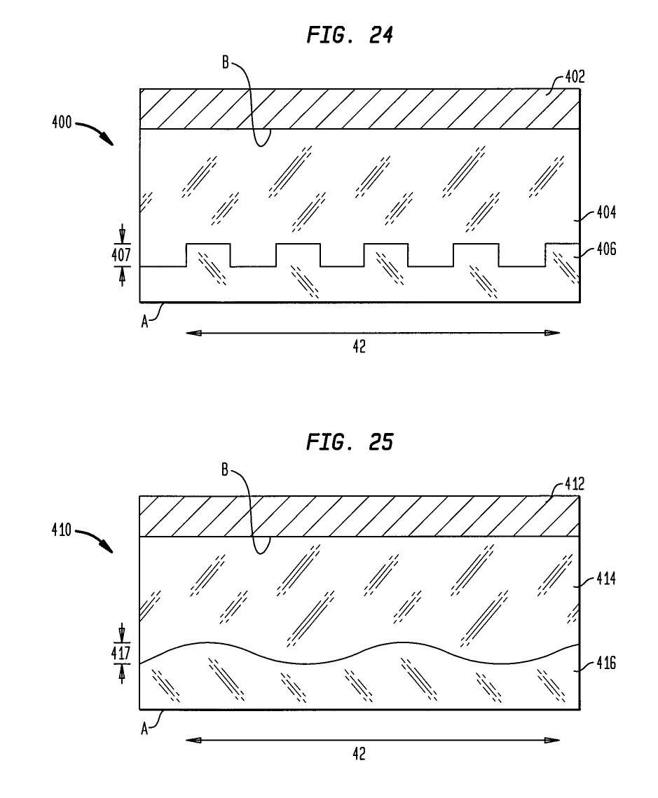

In FIG. 24 and FIG. 25 a treatment device 400, 410 is shown including a transducer 402, 412 and a waveguide 404, 414 comprising a skin contacting element 406, 416. The embodiment of FIG. 24 allows the energy delivered by transducer 402 to be focused into focal regions at different depths below the skin. As illustrated in FIG. 24, the thickness of the skin contacting element 406 varies in a direction parallel to dimension 42. The discontinuous thickness variation 407 of the skin contacting element 404 is designed to focus the energy delivered by transducer 402 to at least two depths. The alternative embodiment of FIG. 25 allows the energy delivered by transducer 412 to be focused into focal regions at a range of depths below the skin. As illustrated in FIG. 25, the thickness of the skin contacting element 414 varies continuously in a direction parallel to dimension 42. The continuous thickness variation 417 of the skin contacting element 414 is designed to focus the energy delivered by transducer 412 to a range of depths below the skin.

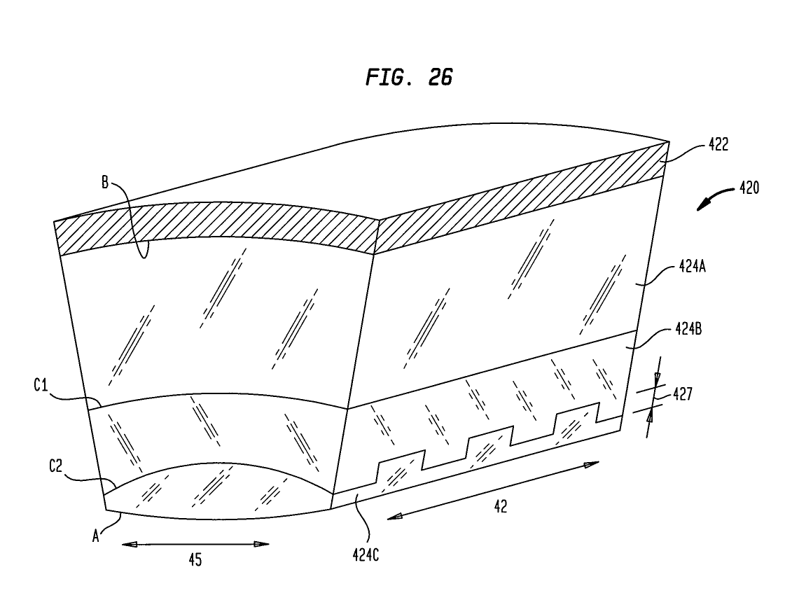

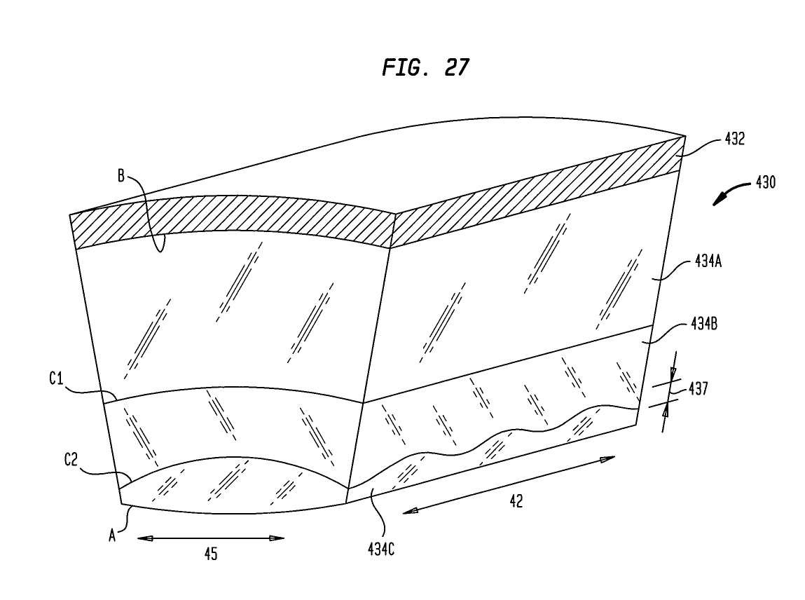

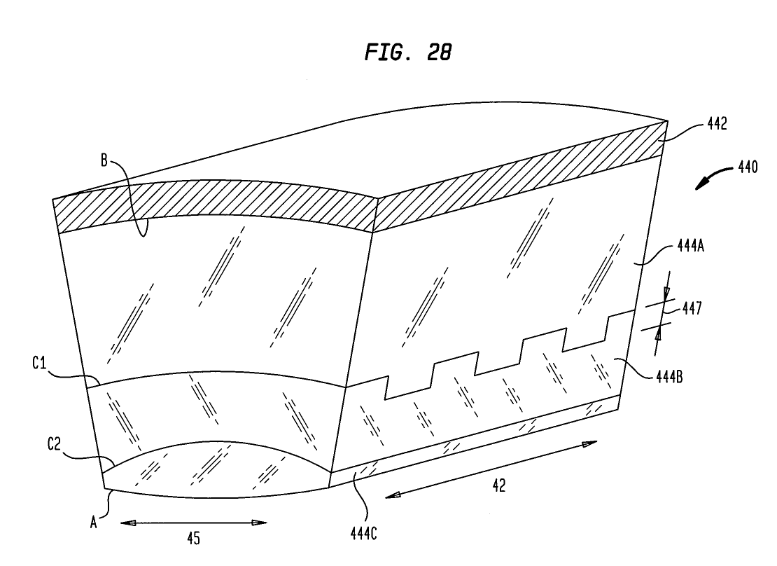

In FIG. 26 and FIG. 27 a treatment device 420, 430 is shown including transducer 422, 432 and a multi-part waveguide 424A-C and 434A-C. One skilled in the art will appreciate that the waveguide can consist of any number of elements, including a single skin contacting element 424C, 434C, to allow the wavefront produced by transducer 422,434 to be adjusted as desired. One skilled in the art will also appreciate that interface C2 can be flat or curved along dimension 45 as necessary for manufacturability or to compensate the distortion in interface A. The embodiment of FIG. 26 allows the energy delivered by transducer 422 to be focused into focal regions at different depths below the skin. As illustrated in FIG. 26, the thickness of the skin contacting element 424C of the multi-part waveguide 424A-C varies in a direction parallel to dimension 42. The discontinuous thickness variation 427 of the skin contacting element 424C shown in FIG. 26 is designed to focus the energy delivered by transducer 422 to at least two different depths.

The alternative embodiment of FIG. 27 allows the energy delivered by transducer 432 to be focused into focal regions at a range of depths below the skin. As illustrated in FIG. 27, the thickness of the skin contacting element 434C of the multi-part waveguide 434A-C varies continuously in a direction parallel to dimension 42. The continuous thickness variation 437 of the skin contacting element 434C is designed to focus the energy delivered by transducer 432 to a range of depths below the skin.

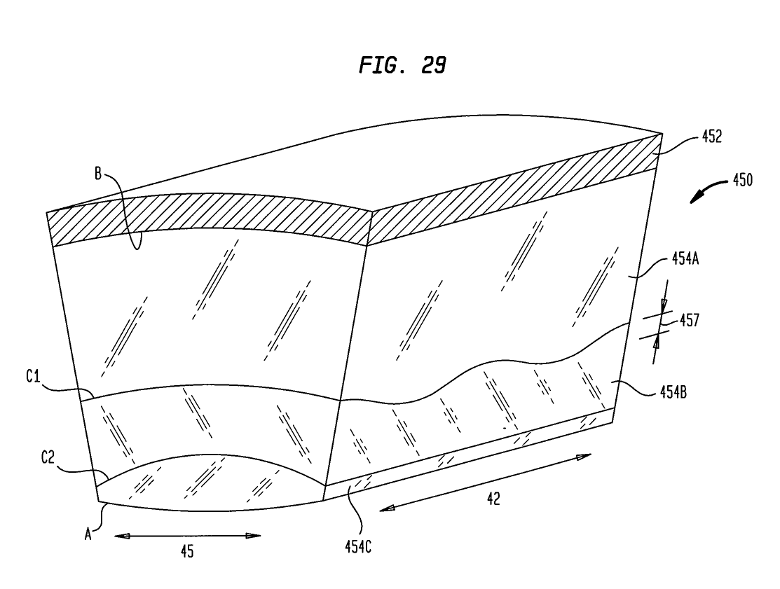

In FIG. 28 and FIG. 29 a treatment device 440, 450 is shown including transducer 442, 452 and a multi-part waveguide 444A-C and 454A-C. One skilled in the art will appreciate that the waveguide can consist of any number of elements allowing the wavefront produced by transducer 442,454 to be adjusted as desired. One skilled in the art will also appreciate that interface Cl can be flat or curved along dimension 45 as necessary for manufacturability or to compensate the distortion in interface A. The embodiment of FIG. 28 allows the energy delivered by transducer 442 to be focused into focal regions at different depths below the skin. As illustrated in FIG. 28, the thickness of the wavefront compensating element 444B of the multi-part waveguide 444A-C varies in a direction parallel to dimension 42. The discontinuous thickness variation 447 of the wavefront compensating element 444B shown in FIG. 28 is designed to focus the energy delivered by transducer 442 to at least two different depths.

The alternative embodiment of FIG. 29 allows the energy delivered by transducer 452 to be focused into focal regions at a range of depths below the skin. As illustrated in FIG. 29, the thickness of the wavefront compensating element 454B of the multi-part waveguide 454A-C varies continuously in a direction parallel to dimension 42. The continuous thickness variation 457 of the wavefront compensating element 454B is designed to focus the energy delivered by transducer 452 to a range of depths below the skin.

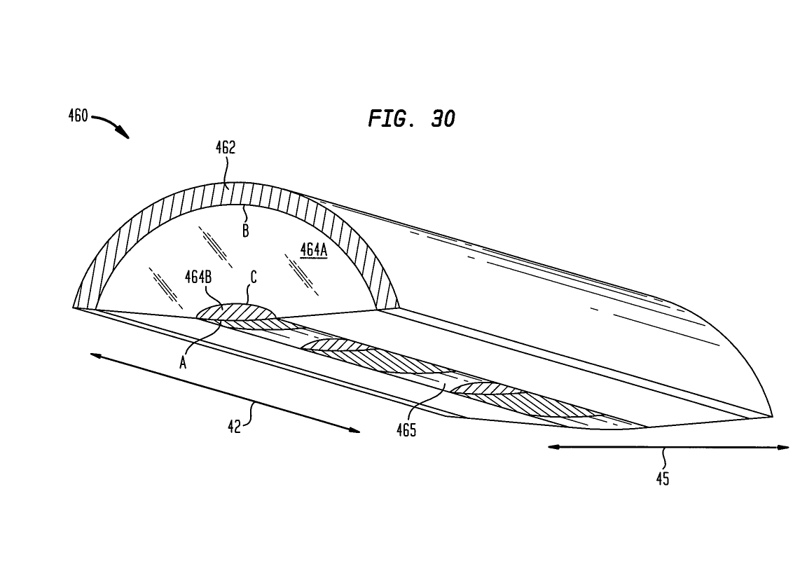

In FIG. 30 a treatment device 460 is shown including transducer 462 and waveguide 424A, 464B. One skilled in the art will appreciate that the waveguide can consist of any number of elements to allow the wavefront produced by transducer 462 to be adjusted as desired. One skilled in the art will also appreciate that interfaces A, B, and C can be flat or curved along dimension 45 as necessary for manufacturability or to compensate the distortion in interface A. The embodiment of FIG. 30 allows the energy delivered by transducer 462 to be focused into focal regions at different depths below the skin. As illustrated in FIG. 30, the thickness of the skin contacting element 464B of the waveguide 424A, 464B varies in a direction parallel to dimension 42 forming recesses 465. During use of the device, recesses 465 may filled with an acoustical coupling medium, such as a biocompatible hydrogel between the surface of the waveguide and the skin. The exemplary stepwise thickness variation of the skin contacting element 464B shown in FIG. 30 is designed to focus the energy delivered by transducer 462 to at least two different depths.

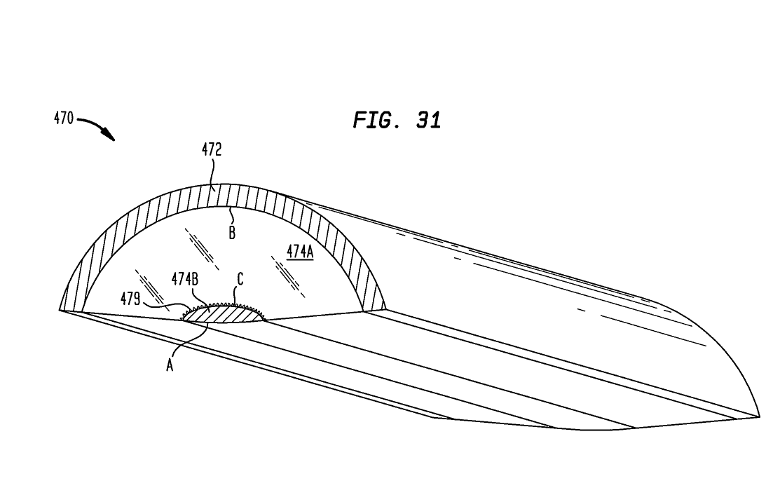

In FIG. 31 a treatment device 470 is shown including transducer 472 and waveguide 474A, 474B. One skilled in the art will appreciate that the waveguide can consist of any number of elements to allow the wavefront produced by transducer 472 to be adjusted as desired. The embodiment of FIG. 31 allows the energy delivered by transducer 472 to be focused into focal regions at different depths below the skin using a diffraction pattern 479. As illustrated in FIG. 31, interface C comprises a diffraction pattern 479. Diffraction pattern 479 may be undular or stepwise. Diffraction pattern 479 preferably comprises a feature height of less than ½ wavelength.

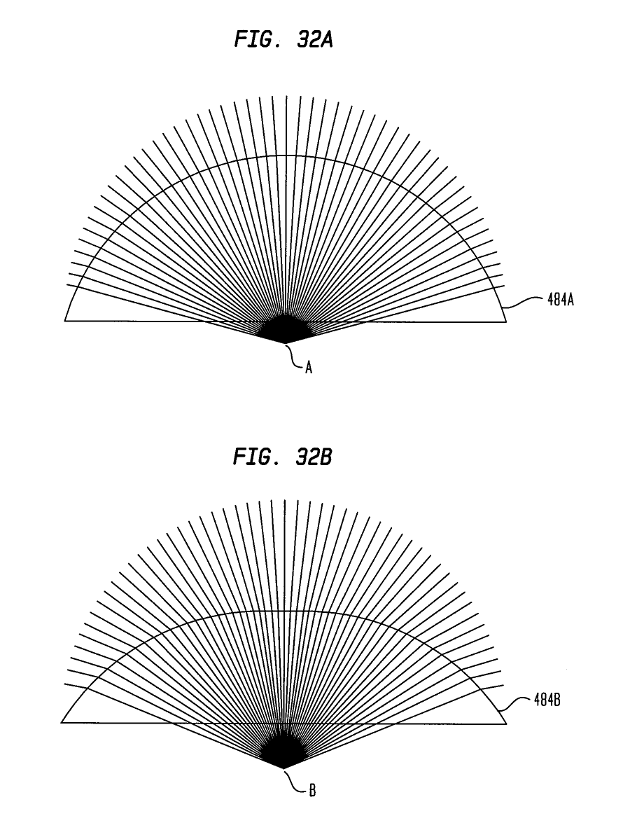

FIG. 32A is an illustration in the form of a simulated ray-tracing analysis showing an acoustic pulse from a waveguide 484A focused at focal point A. FIG. 32B is an illustration in the form of a simulated ray-tracing analysis showing an acoustic pulse from a waveguide 484B focused at focal point B.

In all of the embodiments discussed above, multiple waveguide materials and/or bonding adhesives can be employed and can be designed to minimize cumulative reflection losses of energy from the acoustical wave on its way from the transducer to the target tissue region. These reflection losses can appear at the boundaries between materials of a composite waveguide or between the waveguide and the tissue and/or the transducer and the waveguide. Generally speaking, reflection losses are the result of acoustical impedance mismatch. Various techniques are known in the art to minimize reflection losses including, for example, the use of resonant λ/4 impedance matching layers or alternatively the use of multi-resonant multilayered “broadband” stacks.

It should be noted that the methods of the present invention do not necessarily, following application of ultrasound energy, cause skin wrinkles to be reduced or to disappear immediately. The treatments typically need to be repeated over a long period of time (such as days or months) so that the dermis layer is gently stimulated or irritated to produce the biological response while at the same time avoiding catastrophic damage to the epidermis layer. This has a number of advantages over conventional methods. First, the epidermis is not damaged or is only minimally damaged or effected. Second, the dermis layer is not exposed so the chance of opportunistic infection is reduced. Third, due to the relatively low power levels used and the fact that the epidermis is not catastrophically damaged, the discomfort and pain to the patient compared to conventional methods is considerably reduced.

As noted, the method of the invention using hyperthermia aims to denature a relatively small fraction of the proteins in the dermis, typically less than twenty percent of the proteins. These proteins can be intracellular, extracellular, or also enzymes. Preferably less than ten percent of the proteins in the dermis are denatured and, to be certain that there is much less damage to the cells of the epidermis, no more than approximately five percent of the proteins in the dermis should be denatured.

To further prevent elevation of the temperature or irritation of the epidermis layer of the skin, a cooling device or method can be used. A sapphire tip can be disposed on the ultrasound transducer. Alternatively, water cooling can be used before, during, or after treatment. One skilled in the art will appreciate that there are numerous cooling devices or methods that could be used in conjunction with the invention.

Heating or cooling of the skin can also be used to bring the temperature of the skin to a known state prior to treatment so as to control the dosage of applied ultrasound. This can be significant since denaturation of proteins is dependent on the absolute temperature of the skin and not the relative temperature increase with respect to the starting skin temperature. Heating or cooling of the skin can also be used to take into account patient-to-patient variability such as differing body temperatures to bring all patients to the same state before treatment.

A marker can also be used to delimit treatment areas. The marker can be any kind of suitable marker. For example, a fluorescent gel can be deposited on the skin as the transducer is scanned across the skin. Ink, paint, or disinfectant can also be used. The marker can be visible or can be invisible except when exposed with a suitable light source. A marker allows the user to guide the transducer to produce a spatially uniform ultrasound dosage to ensure uniform stimulation of the dermis and avoid over-treating areas of the skin while under-treating others.

The invention can also reduce other types of defects in skin appearance, such as acne scars and burns, and rejuvenate or refresh skin appearance. This is, as the new connective synthesized in response to the stimulation or irritation of the dermis begins to fill out the dermis, these types of skin defects can become less visible and the skin takes on smoother, refreshed or rejuvenated look.

Having thus described illustrative embodiments of the invention, various alterations, modifications, and improvements will readily occur to those skilled in the art. For example, various alternative acoustic pulse or “shock-wave” generators can be employed in lieu of the above described ultrasound transducers. Such alternative energy generators include piezoelectric, electric spark and laser-triggered pulse forming devices operating on rapid state changes of liquid media or on thermo-elastic expansion. The pulse generated by these devices can exhibit broad frequency domains. Accordingly, the foregoing description is by way of example only and is not intended as limiting. The invention is limited only as defined in the following claims and the equivalents thereto.

What is claimed is:

1. An ultrasound apparatus comprising:
a handpiece adapted for handheld operation,
an acoustic energy generator disposed at least partially in the handpiece; and
a cartridge connected to the handpiece.

2. The apparatus of claim 1 wherein the cartridge comprises a disposable cartridge.

3. The apparatus of claim 1 wherein the cartridge is removable.

4. The apparatus of claim 1 wherein the cartridge contains a fluid.

5. The apparatus of claim 4 wherein the fluid is: an acoustic gel; a coolant; a marker; or a therapeutic agent.

6. The apparatus of claim 1 further comprising a sensor.

7. The apparatus of claim 6 wherein the acoustic energy generator is responsive to an output of said sensor.

8. The apparatus of claim 1 further comprising a control switch.

9. An ultrasound apparatus comprising:
a handpiece adapted for handheld operation,
an acoustic energy emitter disposed at least partially in the handpiece; and
a fluid dispenser connected to the handpiece.

10. The apparatus of claim 9 wherein the fluid dispenser comprises a plurality of fluid dispensing elements.

11. The apparatus of claim 10 wherein the fluid dispensing elements are located proximal to a skin contacting surface and spaced apart from each other.

12. The apparatus of claim 10 wherein each fluid dispensing element comprises a dispenser of an acoustic coupling gel.

13. The apparatus of claim 12 wherein the cartridge is removable.

14. The apparatus of claim 13 wherein the cartridge is removable.

15. The apparatus of claim 12 wherein the cartridge comprises a plurality of fluid reservoirs.

16. The apparatus of claim 9 further comprising a sensor.

17. The apparatus of claim 16 wherein the sensor is an electrical conductivity sensor.

18. The apparatus of claim 16 wherein the sensor is an optical sensor.

19. The apparatus of claim 16 wherein the sensor is an ultrasound receiver.

20. The apparatus of claim 16 wherein the sensor is a movement sensor.

21. The apparatus of claim 16 wherein the fluid dispenser is responsive to an output of said sensor.

22. The apparatus of claim 16 further comprising an alarm responsive to an output of said sensor.

23. A method of dispensing fluid from an ultrasound device comprising the steps of:
dispensing a fluid proximal to a skin contacting surface of an ultrasound device,
sensing a parameter related to the amount of fluid proximal to the skin contacting surface of the ultrasound device; and
dispensing additional fluid proximal to the skin contacting surface of the ultrasound device if the amount of fluid is determined to be low.

24. The method of claim 23 wherein the parameter related to the amount of fluid proximal to the skin contacting surface is movement.

25. The method of claim 23 wherein the fluid is an acoustic coupling gel.

26. The method of claim 23 wherein the parameter related to the amount of fluid proximal to the skin contacting surface is electrical conductivity.

27. The method of claim 23 wherein the parameter related to the amount of fluid proximal to the skin contacting surface is ultrasound reflection.

28. The method of claim 23 wherein the fluid comprises a dye.

29. The method of claim 23 wherein the fluid comprises an infrared dye.

30. The method of claim 23 wherein the parameter related to the amount of fluid proximal to the skin contacting surface is light absorption.
